(19) United States
(12) Patent Application Publication
(10) Pub. No.: US 2016/0260907 A1
(43) Pub. Date: Sep. 8, 2016

(54) ORGANIC ELECTROLUMINESCENT MATERIAL AND ORGANIC ELECTROLUMINESCENT DEVICE

(71) Applicants: BEIJING AGLAIA TECHNOLOGY DEVELOPMENT CO., LTD., Beijing (CN); GUANGDONG AGLAIA OPTOELECTRONIC MATERIALS CO., LTD., Foshan, Guangdong (CN)

(72) Inventors: Kam-hung Low, Foshan (CN); Lei Dai, Beijing (CN); Jinxin Chen, Foshan (CN); Lifei Cai, Beijing (CN)

(73) Assignees: BEIJING AGLAIA TECHNOLOGY DEVELOPMENT CO., LTD., Beijing (CN); GUANGDONG AGLAIA OPTOELECTRONIC MATERIALS CO., LTD ., Foshan, Guangdong (CN)

(21) Appl. No.: 15/035,742

(22) PCT Filed: Nov. 3, 2014
(86) PCT No.: PCT/CN2014/090192

§ 371 Date: May 10, 2016

(30) Foreign Application Priority Data
Nov. 11, 2013 (CN) 201310556630.2
Oct. 27, 2014 (CN) 201410581916.0

(51) Int. Cl. H01L 51/00; C09K 11/02; C07D 221/18; C09K 11/06

(52) U.S. Cl. CPC H01L 51/0072; C09K 11/06; C09K 11/02; C07D 221/18; H01L 51/0052; H01L 51/5016

(57) ABSTRACT

An “organic electroluminescent material and organic electroluminescent device (OED)” with the structure as formula (I) is disclosed herein. The OED possesses high electronic transmission and injection capacity by using the compound having an acenaphtho[1,2-c]pyridine (ANP) group as electronic transport material. It also enhances luminous efficiency and lifetime of the device because of its excellent thermal stability and film-forming properties. At the same time, the OED possesses high triplet energy level and excellent electronic transport capacity by using the compound having the ANP group as the main body of phosphorescence material. It enhances the number of electrons in light-emitting layer and the efficiency of the device effectively.

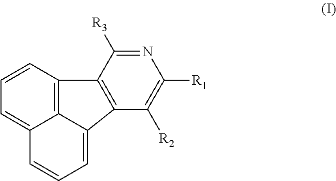

### TECHNICAL FIELD

This invention relates to a new type of organic electroluminescent material. By deposited into thin film through vacuum evaporation, it is used in organic light emitting diodes as an electron transport material and phosphorescence host material. It belongs to the technical field of organic electroluminescent device display.

### BACKGROUND ART

Organic electroluminescent device (OED), as a new type of display technology, has unique advantages such as self-illumination, wide viewing angle, low power consumption, high efficiency, thin, rich colors, fast response, extensive application temperature, low drive voltage, used to make flexible, bendable and transparent display panel and environmental friendliness, etc. Therefore, OED technology can be applied to flat panel displays and new generation of lighting, or can be used as backlight of LCD. Since 1987, Kodak (Tang et al) made sandwich bilayer devices using 8-hydroxyquinoline aluminum (Alq3) as a light emitting layer, triphenylamine derivative as a hole transporting layer through thin-film vacuum evaporation technique. Under 10V driving voltage, the luminance is up to 1000 cd/m² (Tang C. W., Vanslyke S. A. Appl. Phys. Lett. 1987, 51, 913-916). This technological breakthrough has aroused widespread concern in the scientific community and industry, and organic light-emitting research and applications become a hot issue. Subsequently, in 1989, with the invention of host and guest material technology, the luminous efficiency and lifetime of OED is greatly improved. In 1998, Forrest et al found the electroluminescent phosphorescence phenomenon, which broke through the theoretical limit of organic electroluminescent quantum efficiency less than 25%, rising to 100% (Baldo M. A., Forrest S. R. Et al, Nature, 1998, 395, 151-154). Since then, the organic light-emitting entered into a new era, extending the field of research.

A classic three layers of OED comprises a hole transport layer, a light emitting layer and an electron transport layer. Traditionally the electron transport layer of the device adopts Alq₃, which has good film-forming properties and thermal stability, but its strong green light and low electron mobility restricts its industrial applications. Subsequently, some electron transport materials with excellent performance such as 1,3,5-Tris(N-phenylbenzimidazol-2-yl)benzene (TPBI), Bathocuproine (BCP), Bathophenanthroline (Bphen), are also widely used in the OED. The existing material of light-emitting layer can be divided into two categories, namely, fluorescent emitting material and phosphorescent material, which often adopt guest-host doping technology.

CBP (4,4′-bis(9-carbazolyl)-biphenyl) is a highly efficient and high triplet energy level of phosphorescent host material. When CBP is used as the host material, triplet energy can be smoothly transferred to phosphorescent material, to produce efficient red and green light materials. However, these representative host materials are restricted to use because of their thermal stability and short lifetime of manufactured devices.

Although OED has made considerable progress and development after 20 years of development, and organic materials are also in constant development, there are still very few materials with good device efficiency and lifetime and excellent performance and stability that can meet the market demands.

Acenaphtho[1,2-c]pyridine (ANP) has 16π electrons and it is an antiaromatic polycyclic aromatic hydrocarbon compound, composed of two separate conjugate system units (naphthalene and pyridine) by a five-membered ring, and called non-interactive PAHs. The examples about the synthesis of ANP are rarely reported, and ANP and its derivatives have not been used as electroluminescent materials. In this invention, a series of new compounds are invented based on ANP and applied to organic electroluminescent devices.

### SUMMARY OF THE INVENTION

The object of the present invention is to provide synthesis of a novel and efficient organic electron-transport and a phosphorescent host material, the applications in devices, and provide OEDs with high performance and the preparation methods hereof.

The organic electronic material in the present invention has a chemical structural formula (I).

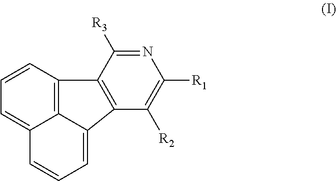

Wherein, R₁-R₃ independently represent hydrogen, deuterium atom, halogen, hydroxy, cyano, nitro, amino, C1-C20 alkyl, C1-C20 alkoxy, C6-C40 aryl containing one or more substituents R or unsubstituted aryl, C6-C40 aromatic hydrocarbon group, C3-C40 aryl containing one or more substituents R or unsubstituted aryl containing one ore more hetero atoms, trialkylsilyl, triaryl silyl, trialkylsilyl containing one or more substituent R or unsubstituted trialkylsilyl, dicarboxylic phosphoroso containing one or more substituent R or unsubstituted diaryl phosphoroso, aryl carbonyl containing one or more substituent R or unsubstituted aryl carbonyl, diaryl amino containing one or more substituent R or unsubstituted diaryl amino, and the hetero atom is B, O, S, N, Se, the substituent R is halogen, hydroxyl, cyano, nitro, amino, C1-C4 alkyl, C1-C4 alkoxy;

Preferably, R₂, R₃ are independently selected from hydrogen, halogen, C1-C8 alkyl, C6-C30 phenyl containing one or more substituent R or unsubstituted phenyl, C10-C30 fused aromatic ring group containing one or more substituent R or unsubstituted one, C6-C20 five- or six-membered heteroaryl containing one or more substituent R or unsubstituted heteroaryl containing one or two hetero atoms, C6-C30 diaromatic amino containing one or more substituent R or unsubstituted diaromatic amino; the substituent R is halogen, cyano, nitro, amino, C1-C4 alkyl, C1-C4 alkoxy, and the hetero atom is O, S, N.

Preferably, R₂, R₃ are independently selected from hydrogen, halogen, C1 -C4 alkyl, phenyl containing one substituent R or unsubstituted phenyl, naphthyl containing one substituent R or unsubstituted naphthyl, carbazolyl containing one substituent R or unsubstituted carbazolyl, five- or six-membered heteroaryl containing one hetero atom, and the substituents R is halogen, amino, C1-C4 alkyl.

The R₂, R₃ can both be hydrogen, C1-C4 alkyl, phenyl, naphthyl, tolyl, thiophene furosemide, furan, pyrrole or pyrazine.

Preferably, wherein R₁ is selected from hydrogen, halogen, C1-C8 alkyl, C6-C20 five-or six-membered heteroaryl containing one or more substituent R or unsubstituted one containing one or more hetero atoms, C10-C20 fused aromatic ring group containing one or more substituent R or unsubstituted one, C6-C30 phenyl containing one or more substituent R or unsubstituted henyl, diphenyl amino, phenyl naphthylamino, triphenyl silyl, diphenylphosphineoxide, phenylcarbonyl or phenylsulfenyl, the substituent R is halogen, cyano, nitro, amino, C1-C4 alkyl, C1-C4 alkoxy, and the heteroatom is O, S, N.

Further preferably, wherein R₁ is selected from hydrogen, halogen, C1-C4 alkyl, C10-C20 carbazolyl containing one substituent R or unsubstituted carbazolyl, C10-C20 fluorenyl containing one substituent R or unsubstituted fluorenyl, naphthyl, phenyl, C6-C10 five- or six-membered heteroaryl containing one or more substituent R or unsubstituted one, and the substituent R is halogen, amino, C1-C4 alkyl.

The five- or six-membered heteroaryl containing one or more hetero atoms is pyrimidinyl, pyridyl, thiazolyl, triazole or triazinyl, the fluorenyl containing one or more substituent R or unsubstituted fluorenyl is 9,9-dimethyl-fluorenyl, 9,9-diphenyl fluorenyl, 9,9-xylyl fluorenyl or spirofluorenyl.

The R₂, R₃ are both phenyl, R₁ is phenyl, bisbiphenyl, naphthyl, carbazolyl substituted by one substituent R, or R₁ is 9,9-dimethyl fluorenyl, 9,9 diphenyl fluorenyl, 9,9-xylyl fluorenyl or spirofluorenyl, and the substituent R is halogen, amino, C1-C4 alkyl.

The following preferred compounds can further illustrate the invention, which should not be considered to limit this invention in any way.

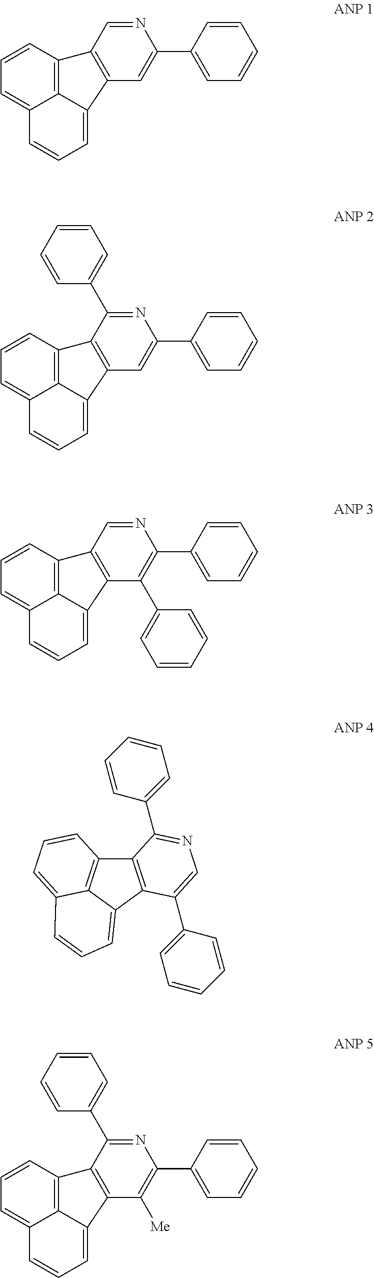 [SMILES: c1ccc(-c2cc3c(cn2)-c2cccc4cccc-3c24)cc1 ; c1ccc(-c2cc3c(c(-c4ccccc4)n2)-c2cccc4cccc-3c24)cc1 ; c1ccc(-c2ncc3c(c2-c2ccccc2)-c2cccc4cccc-3c24)cc1 ; c1ccc(-c2cnc(-c3ccccc3)c3c2-c2cccc4cccc-3c24)cc1 ; Cc1c(-c2ccccc2)nc(-c2ccccc2)c2c1-c1cccc3cccc-2c13]

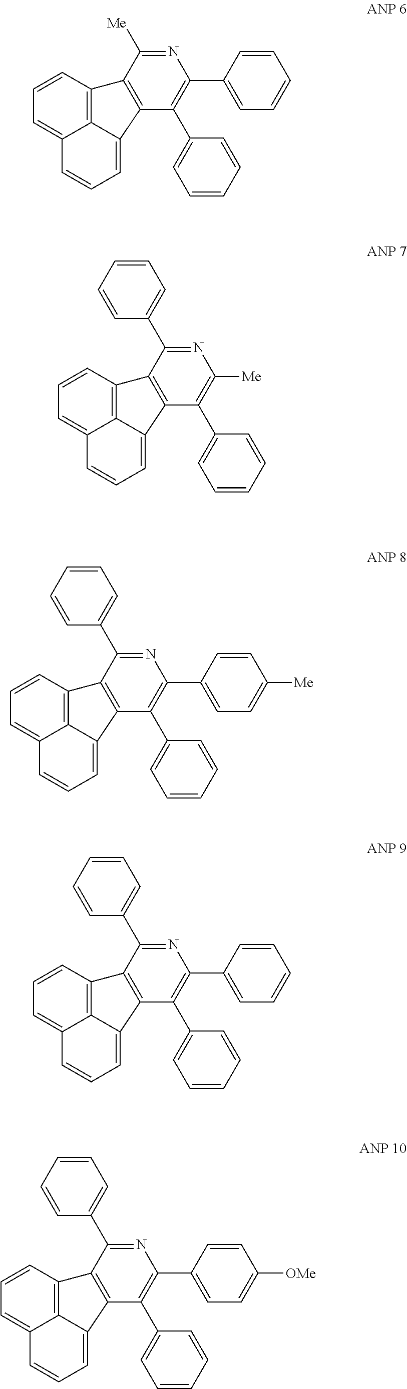 [SMILES: Cc1nc(-c2ccccc2)c(-c2ccccc2)c2c1-c1cccc3cccc-2c13 ; Cc1nc(-c2ccccc2)c2c(c1-c1ccccc1)-c1cccc3cccc-2c13 ; Cc1ccc(-c2nc(-c3ccccc3)c3c(c2-c2ccccc2)-c2cccc4cccc-3c24)cc1 ; c1ccc(-c2nc(-c3ccccc3)c3c(c2-c2ccccc2)-c2cccc4cccc-3c24)cc1 ; COc1ccc(-c2nc(-c3ccccc3)c3c(c2-c2ccccc2)-c2cccc4cccc-3c24)cc1]

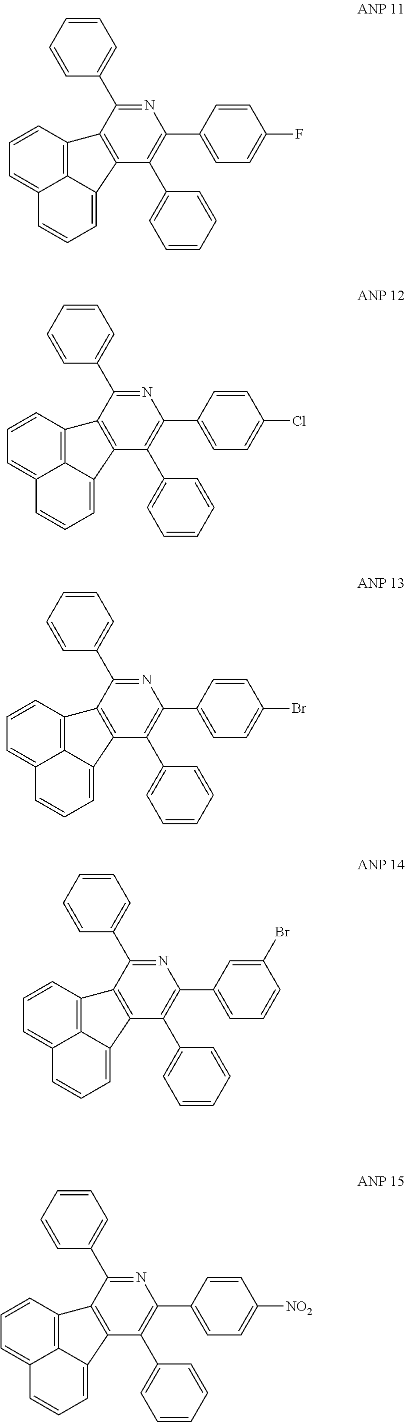 [SMILES: Fc1ccc(-c2nc(-c3ccccc3)c3c(c2-c2ccccc2)-c2cccc4cccc-3c24)cc1 ; Clc1ccc(-c2nc(-c3ccccc3)c3c(c2-c2ccccc2)-c2cccc4cccc-3c24)cc1 ; Brc1ccc(-c2nc(-c3ccccc3)c3c(c2-c2ccccc2)-c2cccc4cccc-3c24)cc1 ; Brc1cccc(-c2nc(-c3ccccc3)c3c(c2-c2ccccc2)-c2cccc4cccc-3c24)c1 ; O=[N+]([O-])c1ccc(-c2nc(-c3ccccc3)c3c(c2-c2ccccc2)-c2cccc4cccc-3c24)cc1]

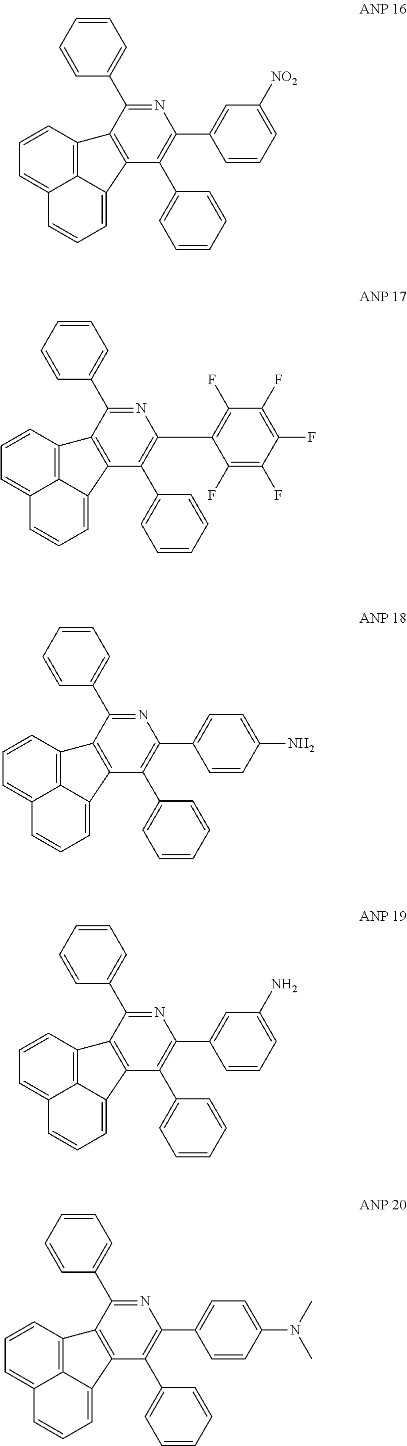 [SMILES: O=[N+]([O-])c1cccc(-c2nc(-c3ccccc3)c3c(c2-c2ccccc2)-c2cccc4cccc-3c24)c1 ; Fc1c(F)c(F)c(-c2nc(-c3ccccc3)c3c(c2-c2ccccc2)-c2cccc4cccc-3c24)c(F)c1F ; Nc1ccc(-c2nc(-c3ccccc3)c3c(c2-c2ccccc2)-c2cccc4cccc-3c24)cc1 ; Nc1cccc(-c2nc(-c3ccccc3)c3c(c2-c2ccccc2)-c2cccc4cccc-3c24)c1 ; CN(C)c1ccc(-c2nc(-c3ccccc3)c3c(c2-c2ccccc2)-c2cccc4cccc-3c24)cc1]

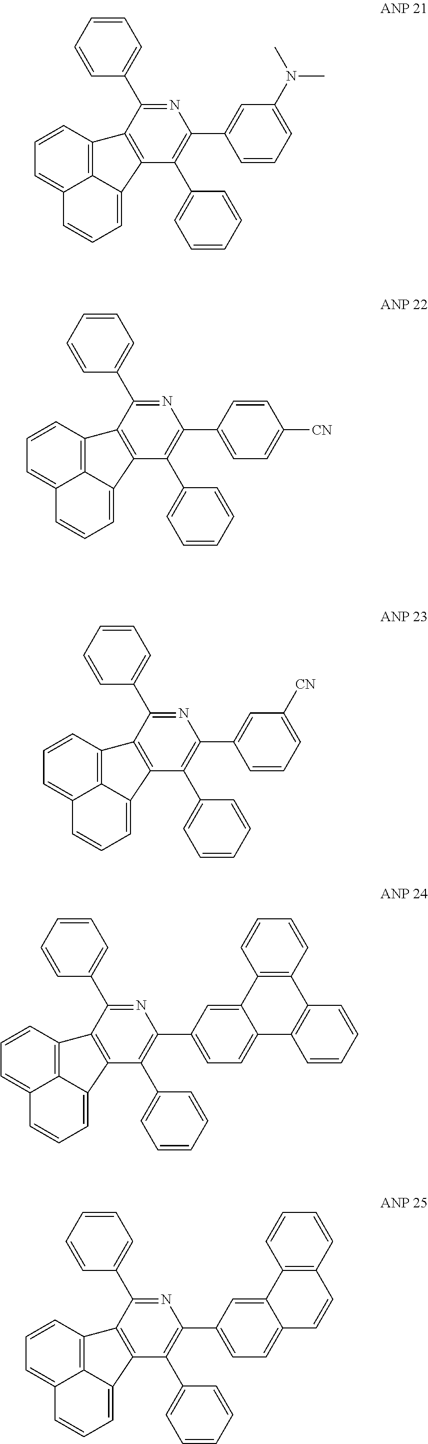 [SMILES: CN(C)c1cccc(-c2nc(-c3ccccc3)c3c(c2-c2ccccc2)-c2cccc4cccc-3c24)c1 ; N#Cc1ccc(-c2nc(-c3ccccc3)c3c(c2-c2ccccc2)-c2cccc4cccc-3c24)cc1 ; N#Cc1cccc(-c2nc(-c3ccccc3)c3c(c2-c2ccccc2)-c2cccc4cccc-3c24)c1 ; c1ccc(-c2nc(-c3ccc4c5ccccc5c5ccccc5c4c3)c(-c3ccccc3)c3c2-c2cccc4cccc-3c24)cc1 ; c1ccc(-c2nc(-c3ccc4ccc5ccccc5c4c3)c(-c3ccccc3)c3c2-c2cccc4cccc-3c24)cc1]

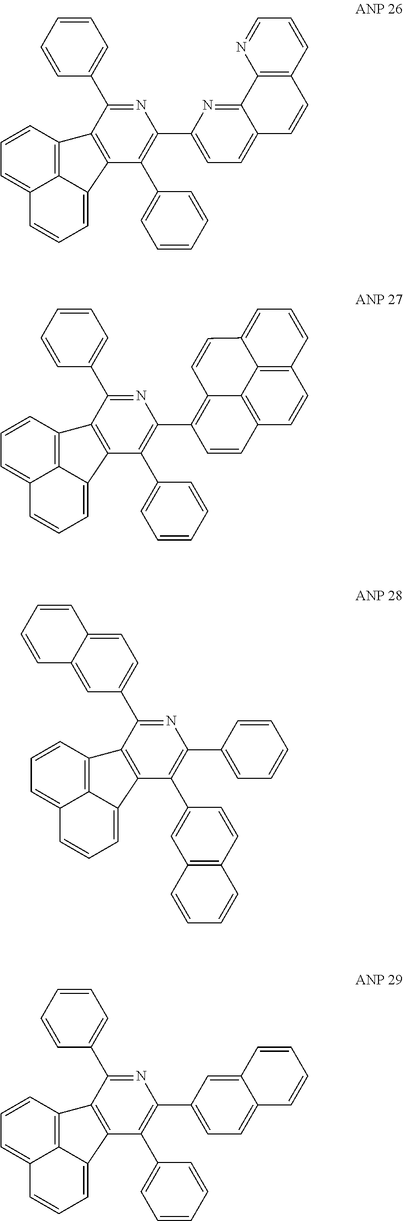 [SMILES: c1ccc(-c2nc(-c3ccc4ccc5cccnc5c4n3)c(-c3ccccc3)c3c2-c2cccc4cccc-3c24)cc1 ; c1ccc(-c2nc(-c3ccc4ccc5cccc6ccc3c4c56)c(-c3ccccc3)c3c2-c2cccc4cccc-3c24)cc1 ; c1ccc(-c2nc(-c3ccc4ccccc4c3)c3c(c2-c2ccc4ccccc4c2)-c2cccc4cccc-3c24)cc1 ; c1ccc(-c2nc(-c3ccc4ccccc4c3)c(-c3ccccc3)c3c2-c2cccc4cccc-3c24)cc1]

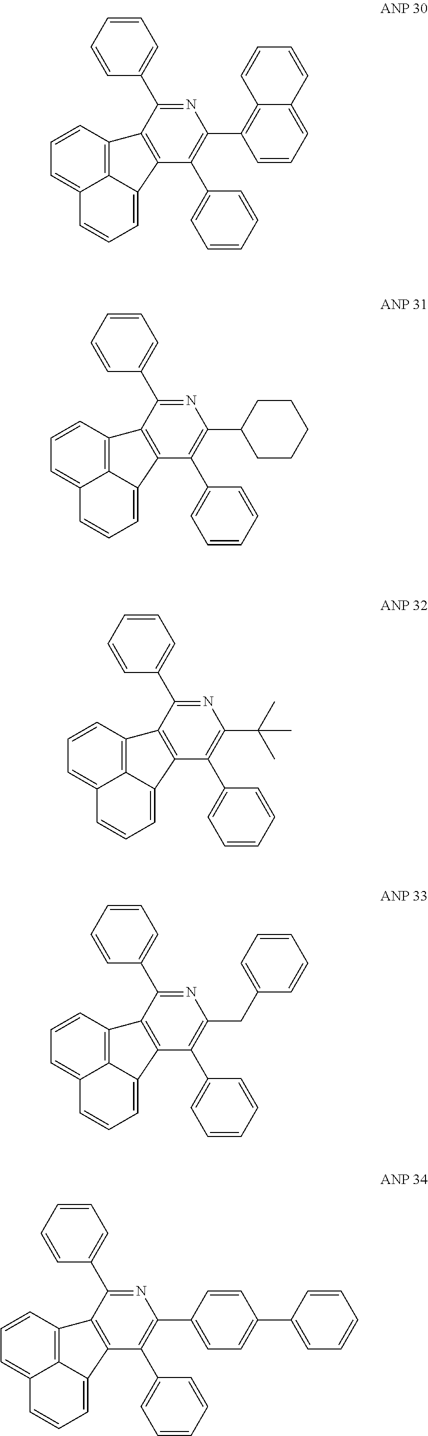 [SMILES: c1ccc(-c2nc(-c3cccc4ccccc34)c(-c3ccccc3)c3c2-c2cccc4cccc-3c24)cc1 ; c1ccc(-c2nc(C3CCCCC3)c(-c3ccccc3)c3c2-c2cccc4cccc-3c24)cc1 ; CC(C)(C)c1nc(-c2ccccc2)c2c(c1-c1ccccc1)-c1cccc3cccc-2c13 ; c1ccc(Cc2nc(-c3ccccc3)c3c(c2-c2ccccc2)-c2cccc4cccc-3c24)cc1 ; c1ccc(-c2ccc(-c3nc(-c4ccccc4)c4c(c3-c3ccccc3)-c3cccc5cccc-4c35)cc2)cc1]

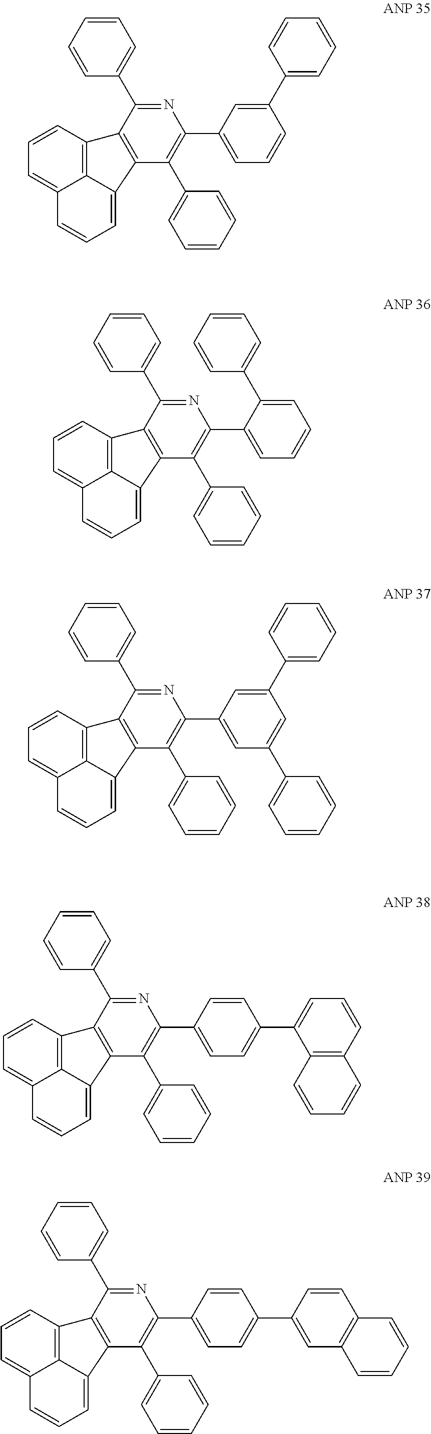 [SMILES: c1ccc(-c2cccc(-c3nc(-c4ccccc4)c4c(c3-c3ccccc3)-c3cccc5cccc-4c35)c2)cc1 ; c1ccc(-c2ccccc2-c2nc(-c3ccccc3)c3c(c2-c2ccccc2)-c2cccc4cccc-3c24)cc1 ; c1ccc(-c2cc(-c3ccccc3)cc(-c3nc(-c4ccccc4)c4c(c3-c3ccccc3)-c3cccc5cccc-4c35)c2)cc1 ; c1ccc(-c2nc(-c3ccc(-c4cccc5ccccc45)cc3)c(-c3ccccc3)c3c2-c2cccc4cccc-3c24)cc1 ; c1ccc(-c2nc(-c3ccc(-c4ccc5ccccc5c4)cc3)c(-c3ccccc3)c3c2-c2cccc4cccc-3c24)cc1]

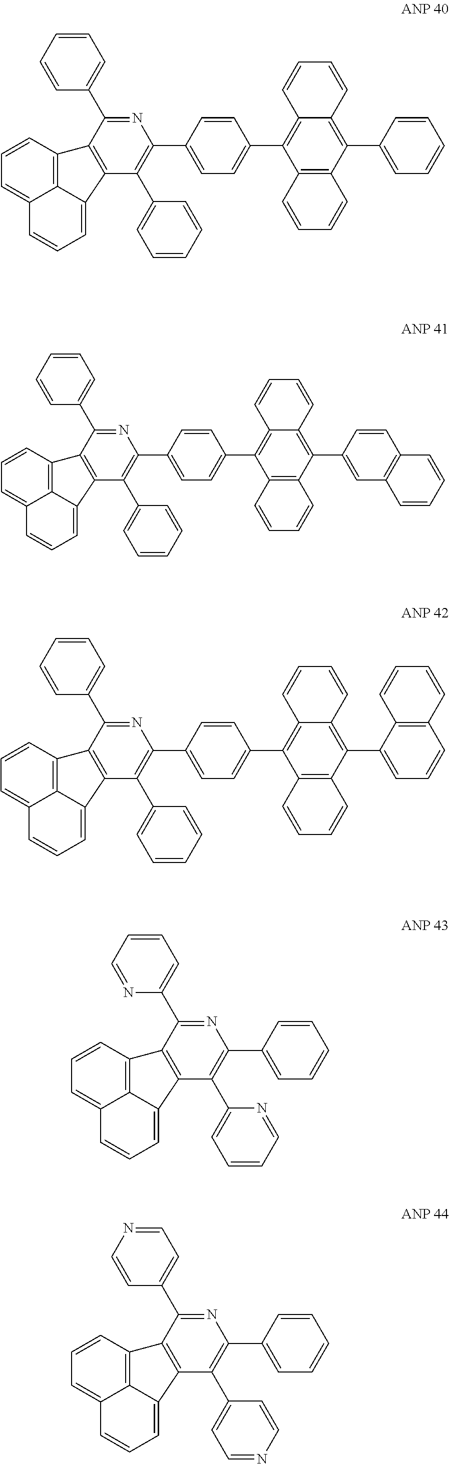 [SMILES: c1ccc(-c2nc(-c3ccc(-c4c5ccccc5c(-c5ccccc5)c5ccccc45)cc3)c(-c3ccccc3)c3c2-c2cccc4cccc-3c24)cc1 ; c1ccc(-c2nc(-c3ccc(-c4c5ccccc5c(-c5ccc6ccccc6c5)c5ccccc45)cc3)c(-c3ccccc3)c3c2-c2cccc4cccc-3c24)cc1 ; c1ccc(-c2nc(-c3ccc(-c4c5ccccc5c(-c5cccc6ccccc56)c5ccccc45)cc3)c(-c3ccccc3)c3c2-c2cccc4cccc-3c24)cc1 ; c1ccc(-c2nc(-c3ccccn3)c3c(c2-c2ccccn2)-c2cccc4cccc-3c24)cc1 ; c1ccc(-c2nc(-c3ccncc3)c3c(c2-c2ccncc2)-c2cccc4cccc-3c24)cc1]

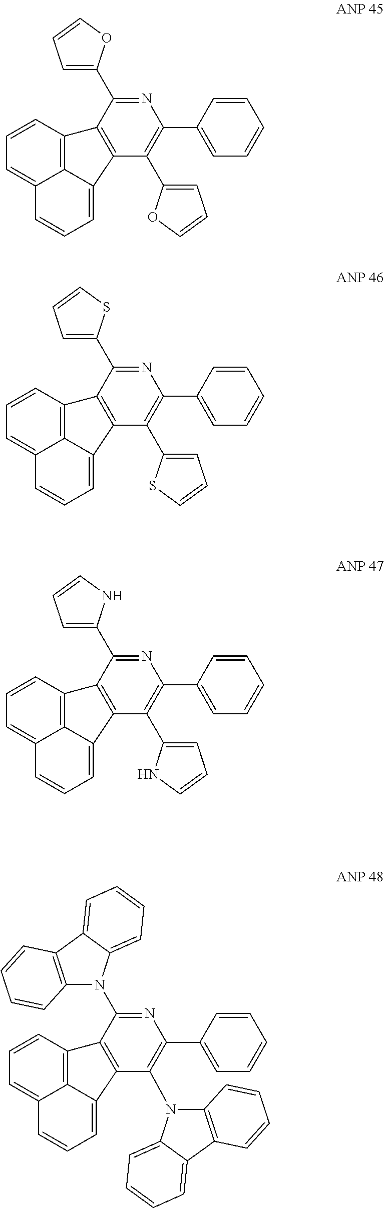 [SMILES: c1ccc(-c2nc(-c3ccco3)c3c(c2-c2ccco2)-c2cccc4cccc-3c24)cc1 ; c1ccc(-c2nc(-c3cccs3)c3c(c2-c2cccs2)-c2cccc4cccc-3c24)cc1 ; c1ccc(-c2nc(-c3ccc[nH]3)c3c(c2-c2ccc[nH]2)-c2cccc4cccc-3c24)cc1 ; c1ccc(-c2nc(-n3c4ccccc4c4ccccc43)c3c(c2-n2c4ccccc4c4ccccc42)-c2cccc4cccc-3c24)cc1]

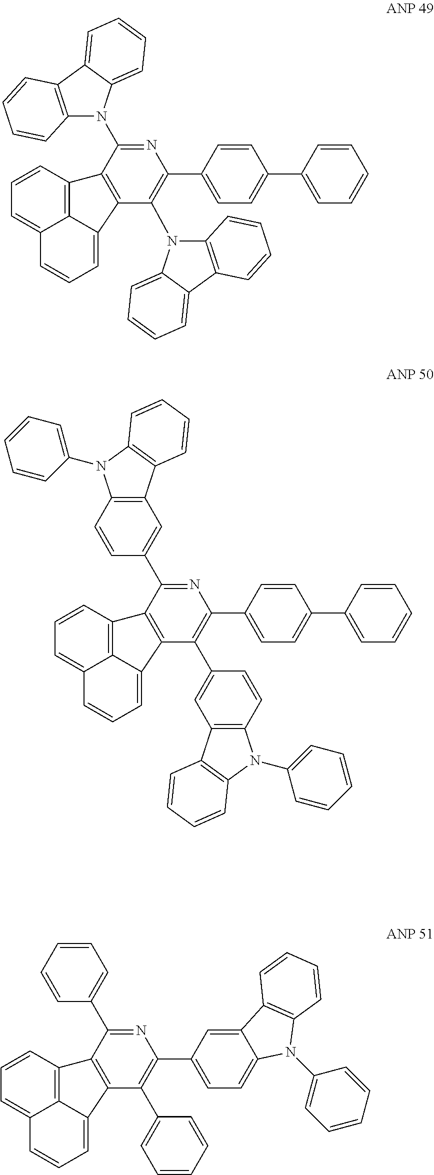 [SMILES: c1ccc(-c2ccc(-c3nc(-n4c5ccccc5c5ccccc54)c4c(c3-n3c5ccccc5c5ccccc53)-c3cccc5cccc-4c35)cc2)cc1 ; c1ccc(-c2ccc(-c3nc(-c4ccc5c(c4)c4ccccc4n5-c4ccccc4)c4c(c3-c3ccc5c(c3)c3ccccc3n5-c3ccccc3)-c3cccc5cccc-4c35)cc2)cc1 ; c1ccc(-c2nc(-c3ccc4c(c3)c3ccccc3n4-c3ccccc3)c(-c3ccccc3)c3c2-c2cccc4cccc-3c24)cc1]

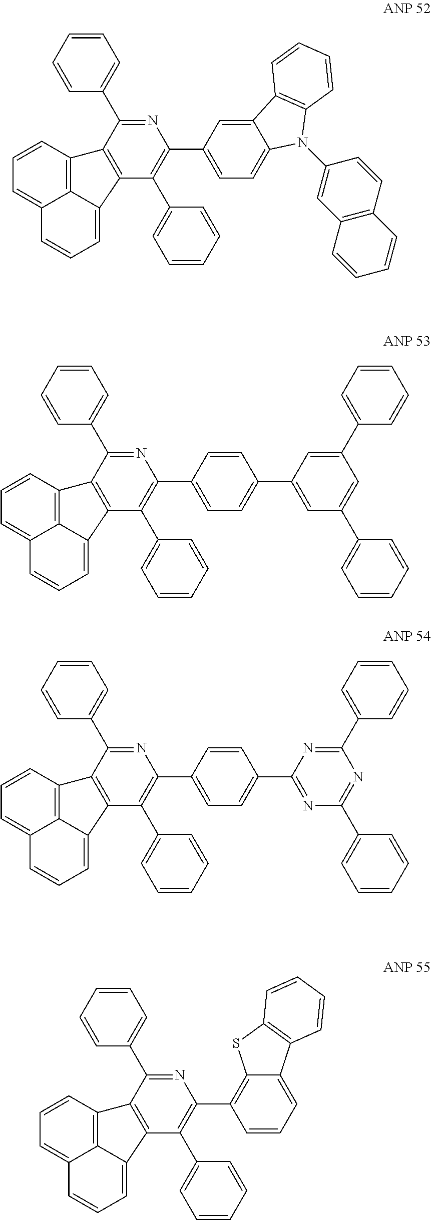 [SMILES: c1ccc(-c2nc(-c3ccc4c(c3)c3ccccc3n4-c3ccc4ccccc4c3)c(-c3ccccc3)c3c2-c2cccc4cccc-3c24)cc1 ; c1ccc(-c2cc(-c3ccccc3)cc(-c3ccc(-c4nc(-c5ccccc5)c5c(c4-c4ccccc4)-c4cccc6cccc-5c46)cc3)c2)cc1 ; c1ccc(-c2nc(-c3ccccc3)nc(-c3ccc(-c4nc(-c5ccccc5)c5c(c4-c4ccccc4)-c4cccc6cccc-5c46)cc3)n2)cc1 ; c1ccc(-c2nc(-c3cccc4c3sc3ccccc34)c(-c3ccccc3)c3c2-c2cccc4cccc-3c24)cc1]

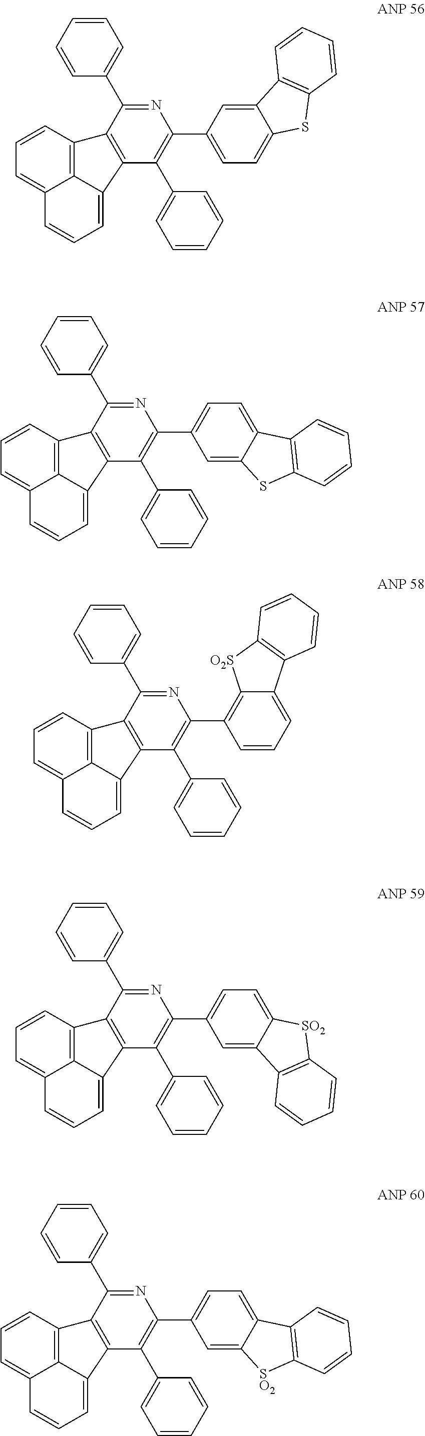 [SMILES: c1ccc(-c2nc(-c3ccc4sc5ccccc5c4c3)c(-c3ccccc3)c3c2-c2cccc4cccc-3c24)cc1 ; c1ccc(-c2nc(-c3ccc4c(c3)sc3ccccc34)c(-c3ccccc3)c3c2-c2cccc4cccc-3c24)cc1 ; O=S1(=O)c2ccccc2-c2cccc(-c3nc(-c4ccccc4)c4c(c3-c3ccccc3)-c3cccc5cccc-4c35)c21 ; O=S1(=O)c2ccccc2-c2cc(-c3nc(-c4ccccc4)c4c(c3-c3ccccc3)-c3cccc5cccc-4c35)ccc21 ; O=S1(=O)c2ccccc2-c2ccc(-c3nc(-c4ccccc4)c4c(c3-c3ccccc3)-c3cccc5cccc-4c35)cc21]

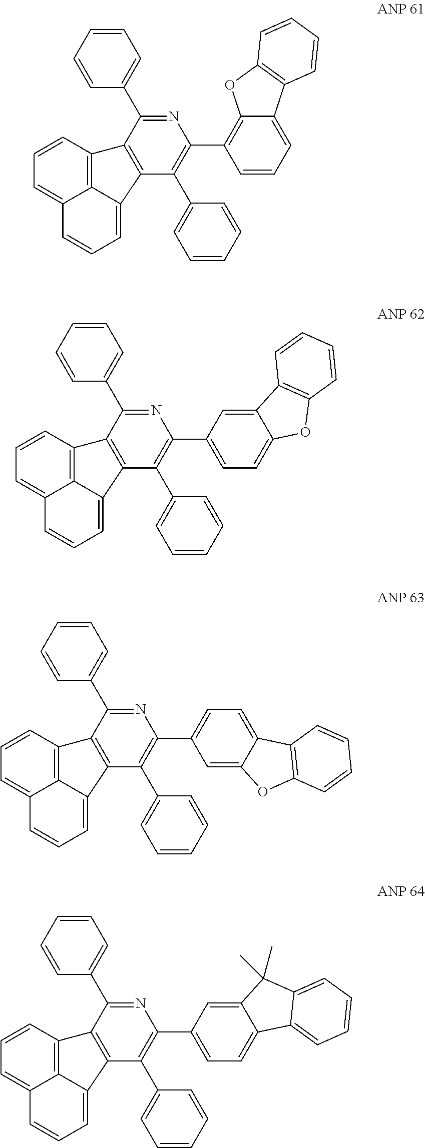 [SMILES: c1ccc(-c2nc(-c3cccc4c3oc3ccccc34)c(-c3ccccc3)c3c2-c2cccc4cccc-3c24)cc1 ; c1ccc(-c2nc(-c3ccc4oc5ccccc5c4c3)c(-c3ccccc3)c3c2-c2cccc4cccc-3c24)cc1 ; c1ccc(-c2nc(-c3ccc4c(c3)oc3ccccc34)c(-c3ccccc3)c3c2-c2cccc4cccc-3c24)cc1 ; CC1(C)c2ccccc2-c2ccc(-c3nc(-c4ccccc4)c4c(c3-c3ccccc3)-c3cccc5cccc-4c35)cc21]

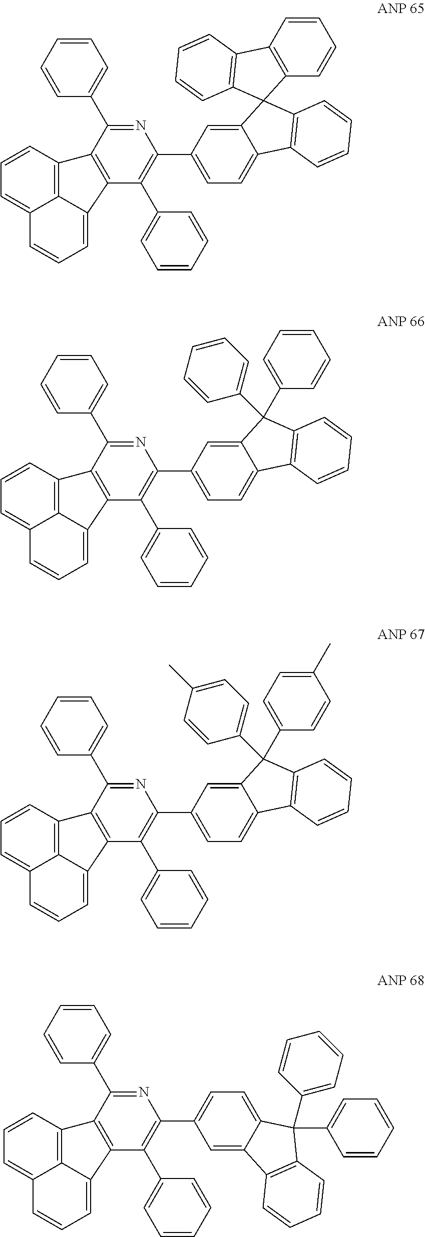 [SMILES: c1ccc(-c2nc(-c3ccc4c(c3)C3(c5ccccc5-c5ccccc53)c3ccccc3-4)c(-c3ccccc3)c3c2-c2cccc4cccc-3c24)cc1 ; c1ccc(-c2nc(-c3ccc4c(c3)C(c3ccccc3)(c3ccccc3)c3ccccc3-4)c(-c3ccccc3)c3c2-c2cccc4cccc-3c24)cc1 ; Cc1ccc(C2(c3ccc(C)cc3)c3ccccc3-c3ccc(-c4nc(-c5ccccc5)c5c(c4-c4ccccc4)-c4cccc6cccc-5c46)cc32)cc1 ; c1ccc(-c2nc(-c3ccc4c(c3)-c3ccccc3C4(c3ccccc3)c3ccccc3)c(-c3ccccc3)c3c2-c2cccc4cccc-3c24)cc1]

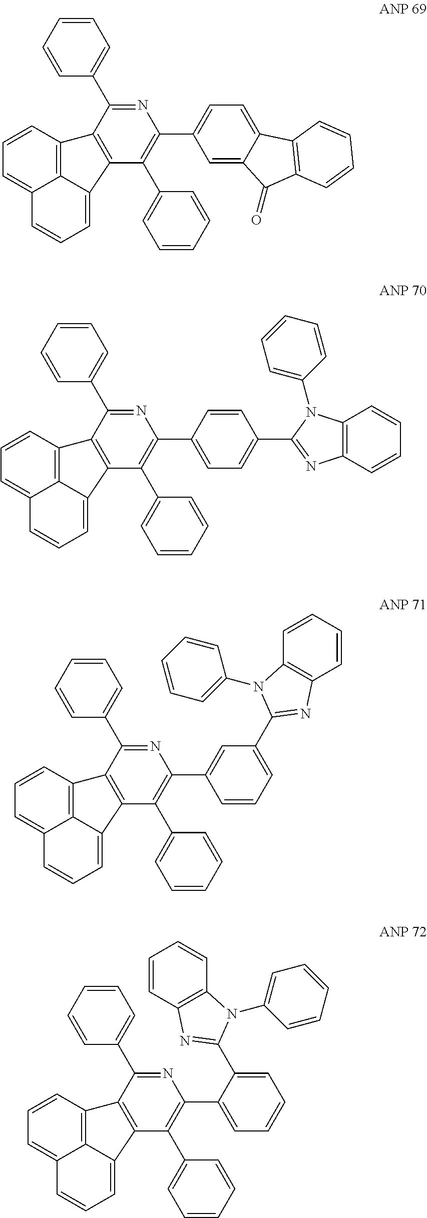 [SMILES: O=C1c2ccccc2-c2ccc(-c3nc(-c4ccccc4)c4c(c3-c3ccccc3)-c3cccc5cccc-4c35)cc21 ; c1ccc(-c2nc(-c3ccc(-c4nc5ccccc5n4-c4ccccc4)cc3)c(-c3ccccc3)c3c2-c2cccc4cccc-3c24)cc1 ; c1ccc(-c2nc(-c3cccc(-c4nc5ccccc5n4-c4ccccc4)c3)c(-c3ccccc3)c3c2-c2cccc4cccc-3c24)cc1 ; c1ccc(-c2nc(-c3ccccc3-c3nc4ccccc4n3-c3ccccc3)c(-c3ccccc3)c3c2-c2cccc4cccc-3c24)cc1]

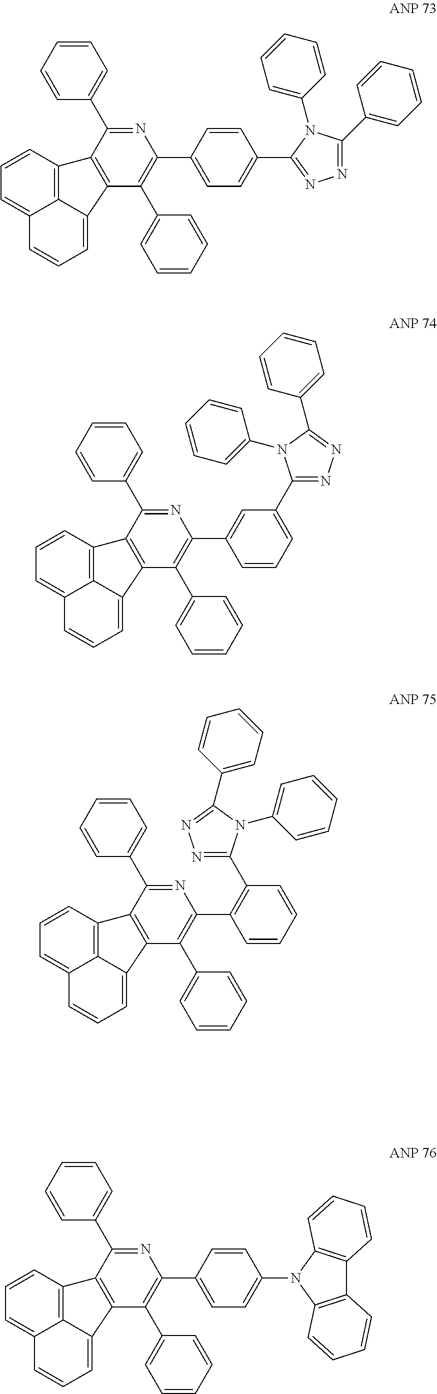 [SMILES: c1ccc(-c2nc(-c3ccc(-c4nnc(-c5ccccc5)n4-c4ccccc4)cc3)c(-c3ccccc3)c3c2-c2cccc4cccc-3c24)cc1 ; c1ccc(-c2nc(-c3cccc(-c4nnc(-c5ccccc5)n4-c4ccccc4)c3)c(-c3ccccc3)c3c2-c2cccc4cccc-3c24)cc1 ; c1ccc(-c2nc(-c3ccccc3-c3nnc(-c4ccccc4)n3-c3ccccc3)c(-c3ccccc3)c3c2-c2cccc4cccc-3c24)cc1 ; c1ccc(-c2nc(-c3ccc(-n4c5ccccc5c5ccccc54)cc3)c(-c3ccccc3)c3c2-c2cccc4cccc-3c24)cc1]

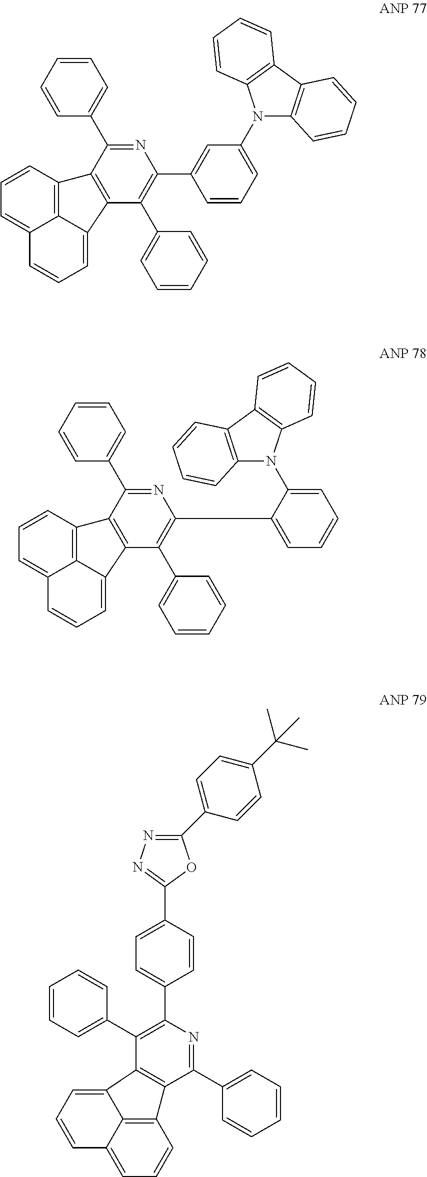 [SMILES: c1ccc(-c2nc(-c3cccc(-n4c5ccccc5c5ccccc54)c3)c(-c3ccccc3)c3c2-c2cccc4cccc-3c24)cc1 ; c1ccc(-c2nc(-c3ccccc3-n3c4ccccc4c4ccccc43)c(-c3ccccc3)c3c2-c2cccc4cccc-3c24)cc1 ; CC(C)(C)c1ccc(-c2nnc(-c3ccc(-c4nc(-c5ccccc5)c5c(c4-c4ccccc4)-c4cccc6cccc-5c46)cc3)o2)cc1]

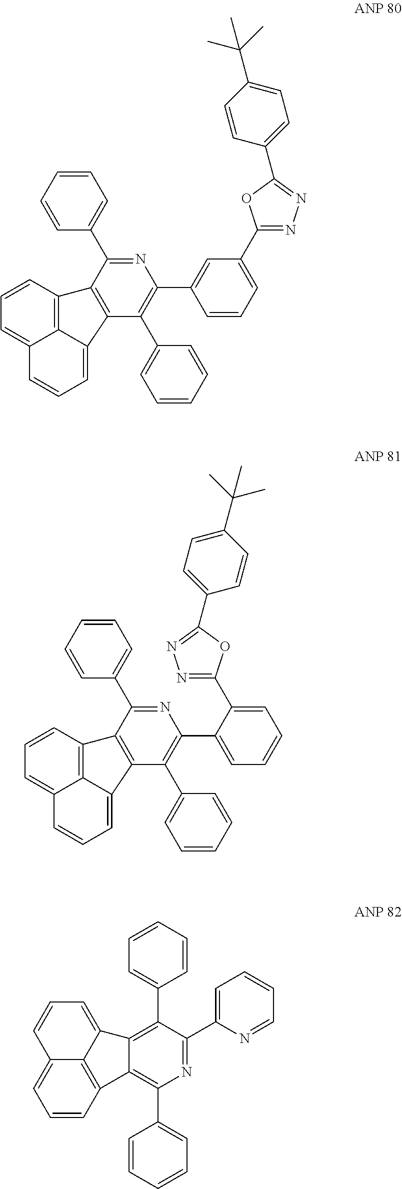 [SMILES: CC(C)(C)c1ccc(-c2nnc(-c3cccc(-c4nc(-c5ccccc5)c5c(c4-c4ccccc4)-c4cccc6cccc-5c46)c3)o2)cc1 ; CC(C)(C)c1ccc(-c2nnc(-c3ccccc3-c3nc(-c4ccccc4)c4c(c3-c3ccccc3)-c3cccc5cccc-4c35)o2)cc1 ; c1ccc(-c2nc(-c3ccccn3)c(-c3ccccc3)c3c2-c2cccc4cccc-3c24)cc1]

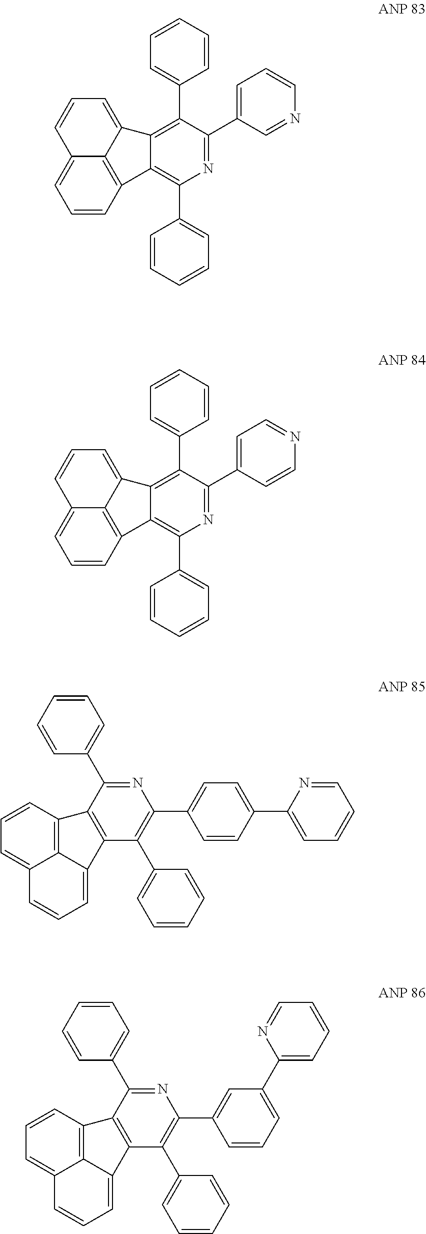 [SMILES: c1ccc(-c2nc(-c3cccnc3)c(-c3ccccc3)c3c2-c2cccc4cccc-3c24)cc1 ; c1ccc(-c2nc(-c3ccncc3)c(-c3ccccc3)c3c2-c2cccc4cccc-3c24)cc1 ; c1ccc(-c2nc(-c3ccc(-c4ccccn4)cc3)c(-c3ccccc3)c3c2-c2cccc4cccc-3c24)cc1 ; c1ccc(-c2nc(-c3cccc(-c4ccccn4)c3)c(-c3ccccc3)c3c2-c2cccc4cccc-3c24)cc1]

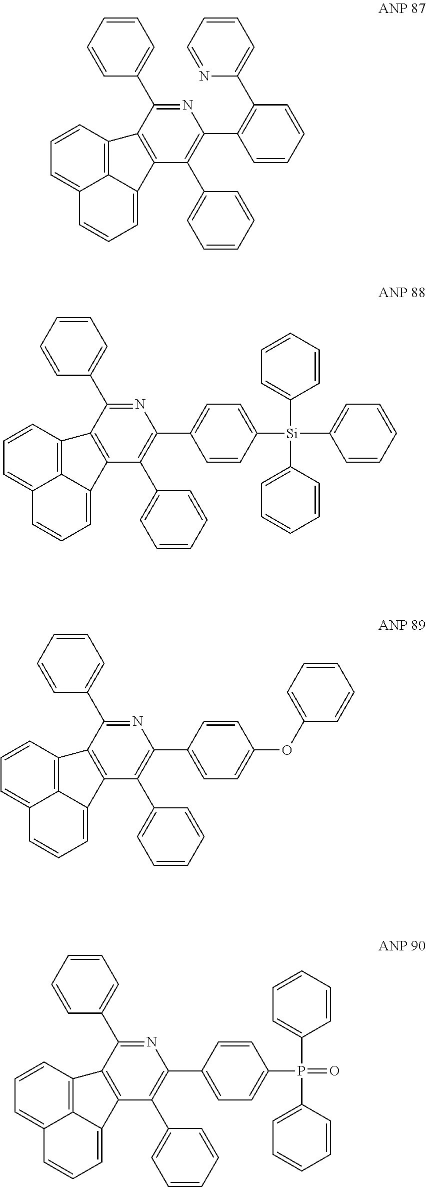 [SMILES: c1ccc(-c2nc(-c3ccccc3-c3ccccn3)c(-c3ccccc3)c3c2-c2cccc4cccc-3c24)cc1 ; c1ccc(-c2nc(-c3ccc([Si](c4ccccc4)(c4ccccc4)c4ccccc4)cc3)c(-c3ccccc3)c3c2-c2cccc4cccc-3c24)cc1 ; c1ccc(Oc2ccc(-c3nc(-c4ccccc4)c4c(c3-c3ccccc3)-c3cccc5cccc-4c35)cc2)cc1 ; O=P(c1ccccc1)(c1ccccc1)c1ccc(-c2nc(-c3ccccc3)c3c(c2-c2ccccc2)-c2cccc4cccc-3c24)cc1]

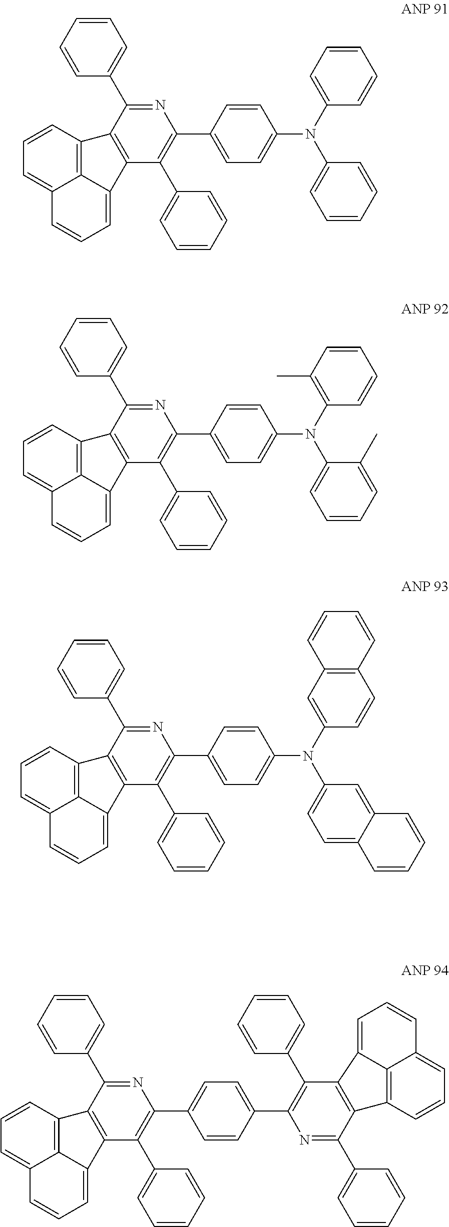 [SMILES: c1ccc(-c2nc(-c3ccc(N(c4ccccc4)c4ccccc4)cc3)c(-c3ccccc3)c3c2-c2cccc4cccc-3c24)cc1 ; Cc1ccccc1N(c1ccc(-c2nc(-c3ccccc3)c3c(c2-c2ccccc2)-c2cccc4cccc-3c24)cc1)c1ccccc1C ; c1ccc(-c2nc(-c3ccc(N(c4ccc5ccccc5c4)c4ccc5ccccc5c4)cc3)c(-c3ccccc3)c3c2-c2cccc4cccc-3c24)cc1 ; c1ccc(-c2nc(-c3ccc(-c4nc(-c5ccccc5)c5c(c4-c4ccccc4)-c4cccc6cccc-5c46)cc3)c(-c3ccccc3)c3c2-c2cccc4cccc-3c24)cc1]

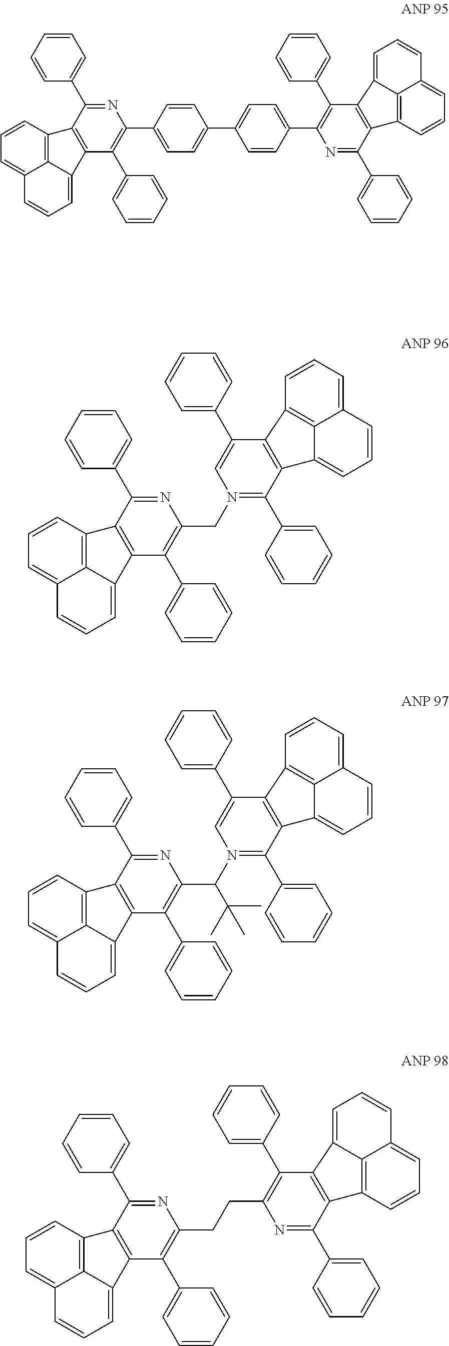

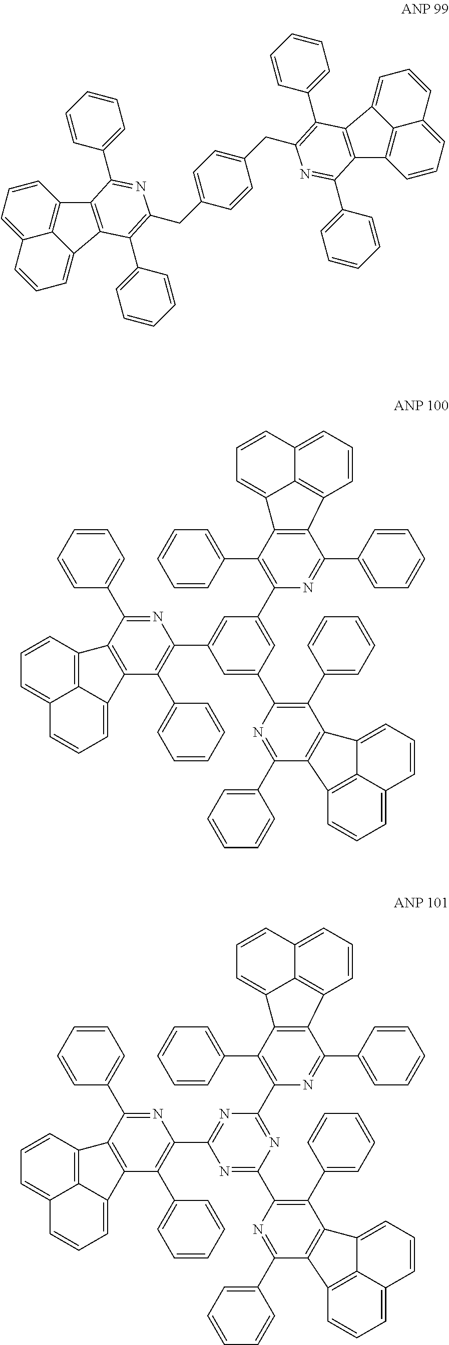 [SMILES: c1ccc(-c2nc(Cc3ccc(Cc4nc(-c5ccccc5)c5c(c4-c4ccccc4)-c4cccc6cccc-5c46)cc3)c(-c3ccccc3)c3c2-c2cccc4cccc-3c24)cc1 ; c1ccc(-c2nc(-c3cc(-c4nc(-c5ccccc5)c5c(c4-c4ccccc4)-c4cccc6cccc-5c46)cc(-c4nc(-c5ccccc5)c5c(c4-c4ccccc4)-c4cccc6cccc-5c46)c3)c(-c3ccccc3)c3c2-c2cccc4cccc-3c24)cc1 ; c1ccc(-c2nc(-c3nc(-c4nc(-c5ccccc5)c5c(c4-c4ccccc4)-c4cccc6cccc-5c46)nc(-c4nc(-c5ccccc5)c5c(c4-c4ccccc4)-c4cccc6cccc-5c46)n3)c(-c3ccccc3)c3c2-c2cccc4cccc-3c24)cc1]

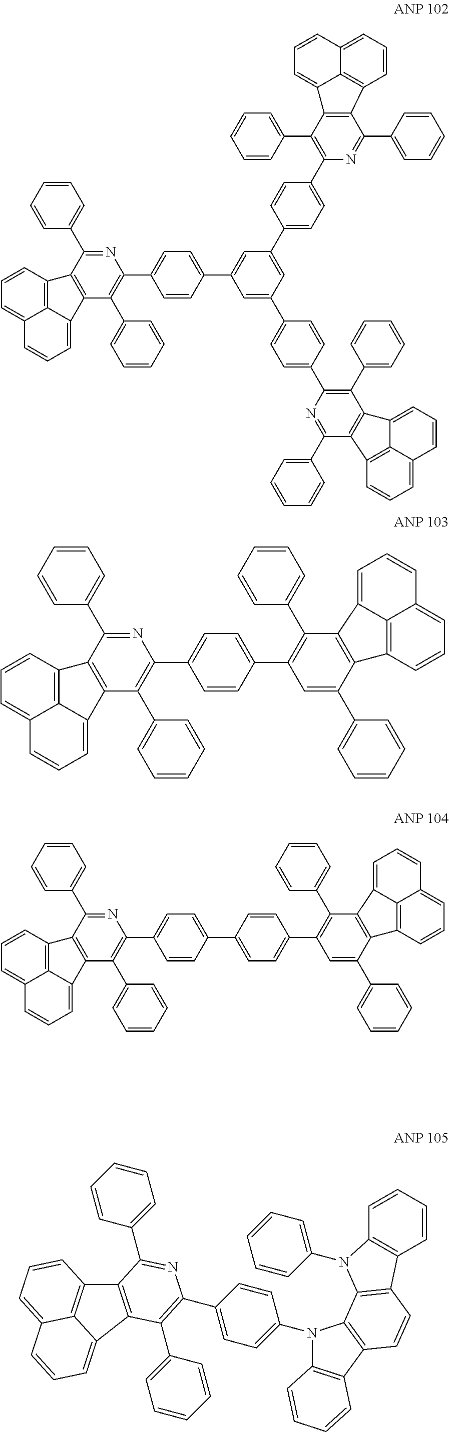

The above method for preparing organic electroluminescent materials is as follows:

(1) Prepare

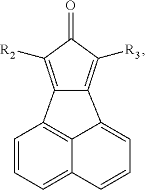

(2) react with R₁—CN for 40-50 hours under the temperature of 250-300° C. with the protection of nitrogen gas.

The reaction in step (2) is to mix the raw materials for direct heating under the protection of nitrogen gas.

The reaction in step (2) is to heat for reflux for 40-50 hours by adding solvent diphenyl ether.

The step (2) further includes recrystallization purification: the recrystallization is to recrystallize and purify with dichloromethane-acetone mixed solvent.

Further the silica gel column purification steps and petroleum ether eluting are included before the recrystallization.

The method in the step (1): reflux the acenaphthequinone and

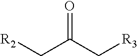

at 70-100° C. under the condition of nitrogen gas and strong alkaline condition.

The strong alkaline condition is to add sodium hydroxide or potassium hydroxide in solution, and the solvent of the reflux solution is ethanol.

The target compound in the invention is a new, efficient organic electron-transport or phosphorescent host material, which is used in high-performance OED. The OED in the invention comprises a substrate, an anode layer formed on the substrate, a hole injection layer, a hole transport layer, a light emitting layer, an electron transport layer and an electron injection layer evaporated sequentially in the anode layer as well as an anode and a cathode.

The light-emitting layer may be a fluorescent light-emitting layer or a red phosphorescent light emitting layer.

In one embodiment of OED in the present invention, the compounds in the invention are used as an electron transporting material;

In another embodiment of OED in the present invention, the above compounds are used as phosphorescent host material, and the guest material is preferably an organic iridium compound and an organic platinum compound;

The OED in the present invention adopts the above compounds as a phosphorescent host material, and adopts the above compounds as an electron transport layer.

The OED possesses high electronic transmission and injection capacity by using the compound having an ANP group as electronic transport material. It also enhances luminous efficiency and lifetime of the device because of its excellent thermal stability and film-forming properties. At the same time, the OED possesses high triplet energy level and excellent electronic transport capacity by using the compound having the ANP group as the main body of phosphorescence material. It enhances the number of electrons in light-emitting layer and the efficiency of the device effectively.

### BRIEF DESCRIPTION OF THE DRAWINGS

FIG. 1 is a structural chart of the device, of which, 10 denotes a glass substrate, 20 denotes an anode, 30 denotes hole injection layer, 40 denotes hole transport layer, 50 denotes light emitting layer, 60 denotes electron transport layer, 70 denotes electron injection layer, 80 denotes cathode.

FIG. 2 is the ESI-MS diagram of ANP 8,

FIG. 3 is the MALDI-TOF-MS diagram of ANP 34,

FIG. 4 is ESI-MS diagram of ANP64,

FIG. 5 is the ¹H NMR diagram of ANP34,

FIG. 6 is the ¹H NMR diagram of ANP64,

FIG. 7 is ¹³C NMR diagram of ANP64,

FIG. 8 is the V-J curves of devices 3 (round), 4 (triangle), and 5 (square).

### DETAILED DESCRIPTIONS OF THE PREFERRED EMBODIMENTS

In the following, the invention is described in details by combining the following embodiments, but it should not be considered to limit the invention.

The raw materials used below are commercially available materials.

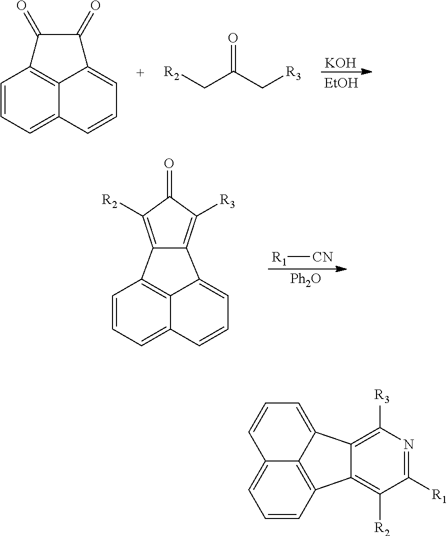

### Embodiment 1: Synthesis of Compound ANP 8

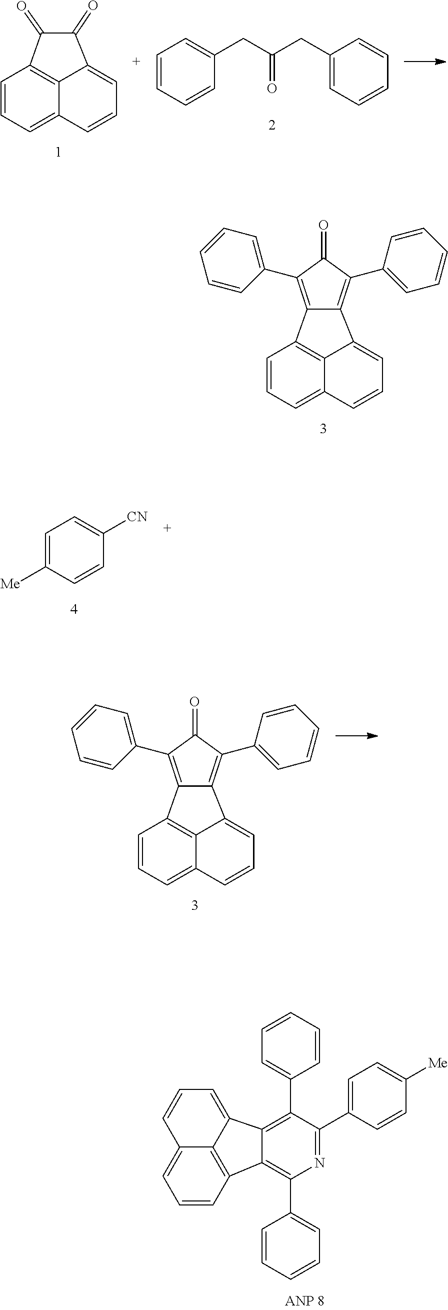

### Synthesis of Intermediate 3

Add anthraquinone (84 g, 0.46 mol), 1,3-diphenyl acetone (72.8 g, 0.34 mol), 600 ml ethanol, 56 g potassium hydroxide to a four-necked flask, to stir and introduce nitrogen for reflux 2 hours, cool down to room temperature, filter; rinse the filter cake by ethanol twice, to get 130 g of black solid, with a yield of 91%.

### Synthesis of Compound ANP 8

Under the nitrogen gas condition, mix the intermediate 3 (3.56 g, 10 mmol) and intermediate 4 (4.69 g, 40 mmol), heat them to reflux 48 hours (external temperature of 280° C.). Cool down the resulting brown solution to get the brown solid. By using petroleum ether as eluant to pass the silica gel columns, crystallize from dichloromethane-acetone to get 0.23 g of ANP 8 white crystal, with a yield of 5%.

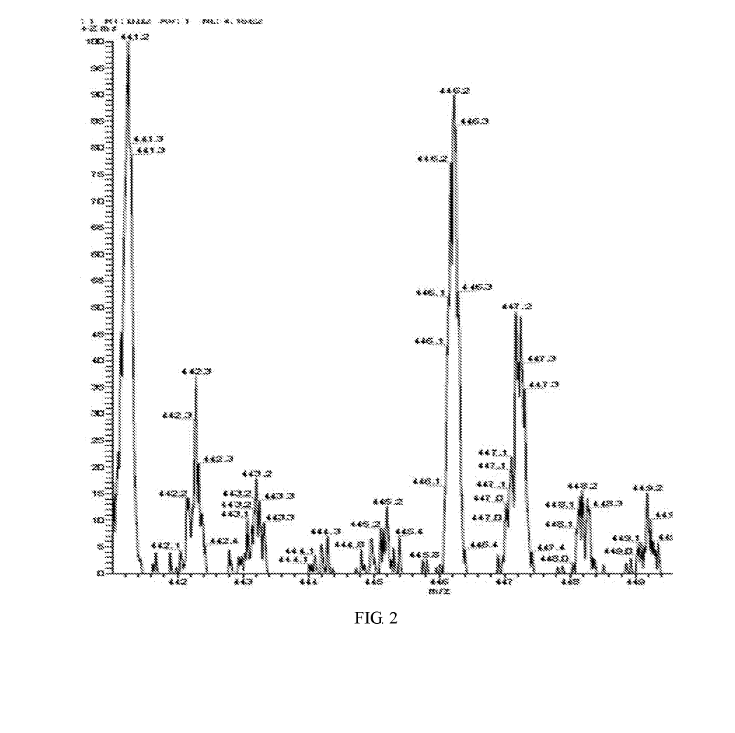

ESI-MS m/s value C₃₄H₂₃N: 445.18, actual measurement value [M⁺]: 446.18, as shown in FIG. 2.

### Embodiment 2: Synthesis of Compound ANP 34

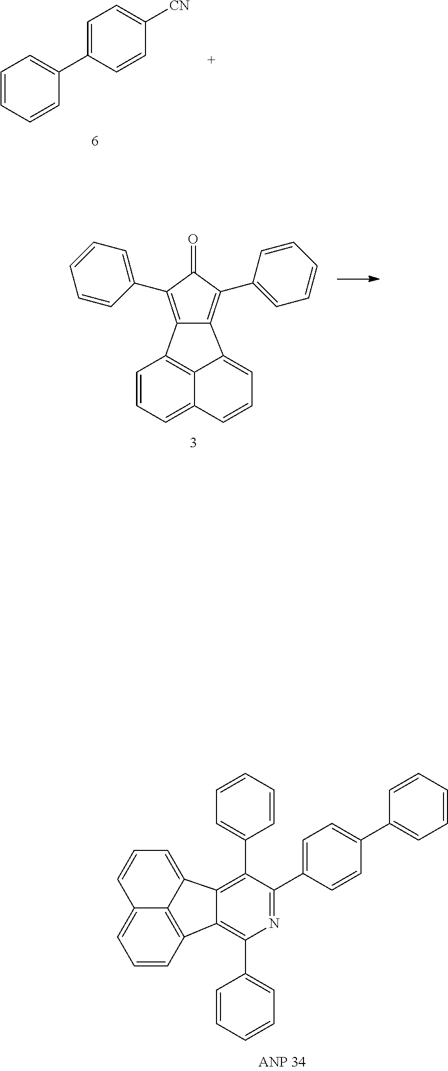 [SMILES: N#Cc1ccc(-c2ccccc2)cc1 ; O=C1C(c2ccccc2)=C2C(=C1c1ccccc1)c1cccc3cccc2c13 ; c1ccc(-c2ccc(-c3nc(-c4ccccc4)c4c(c3-c3ccccc3)-c3cccc5cccc-4c35)cc2)cc1]

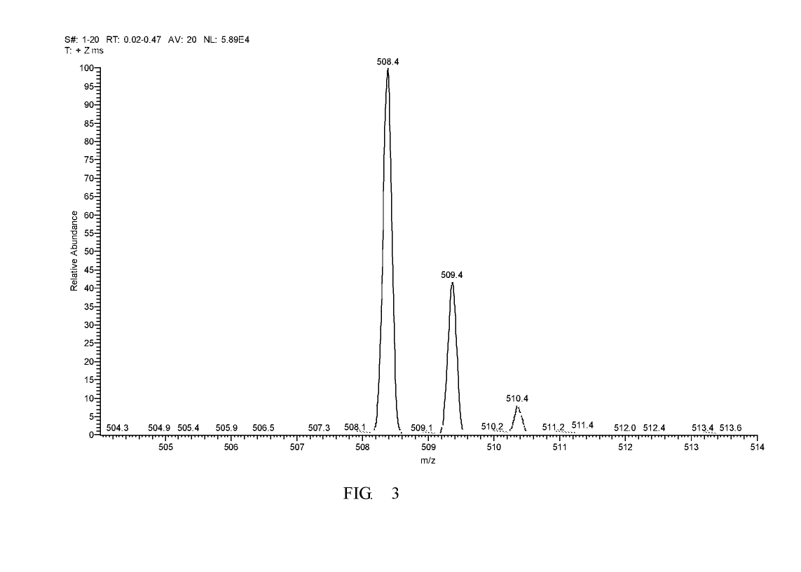

Under the nitrogen gas condition, mix the intermediate 3 (3.56 g, 10 mmol) and intermediate 6 (7.17 g, 40 mmol), heat them to reflux 48 hours (external temperature of 280° C.). Cool down the resulting brown solution to get the brown solid. By using petroleum ether as eluant to pass the silica gel columns, crystallize from dichloromethane-acetone to get 1.37 g of ANP 34 pale yellow crystal, with a yield of 27%. ¹H NMR (400 MHz, CDCl₃, δ): 7.98-7.95 (m, 2 H), 7.90-7.82 (m, 2 H), 7.65-7.31 (m, 20 H), 6.93-6.89 (d, 1 H), as shown in FIG. 5. MALDI-TOF-MS m/s value C₃₉H₂₅N: 507.20, actual measurement value [M+H]⁺: 508.50, as shown in FIG. 3.

### Embodiment 3: Synthesis of Compound ANP 64

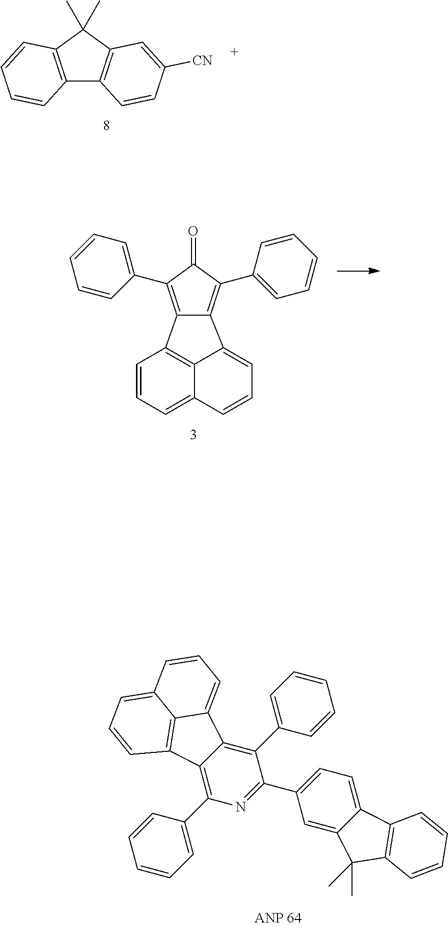 [SMILES: CC1(C)c2ccccc2-c2ccc(C#N)cc21 ; O=C1C(c2ccccc2)=C2C(=C1c1ccccc1)c1cccc3cccc2c13 ; CC1(C)c2ccccc2-c2ccc(-c3nc(-c4ccccc4)c4c(c3-c3ccccc3)-c3cccc5cccc-4c35)cc21]

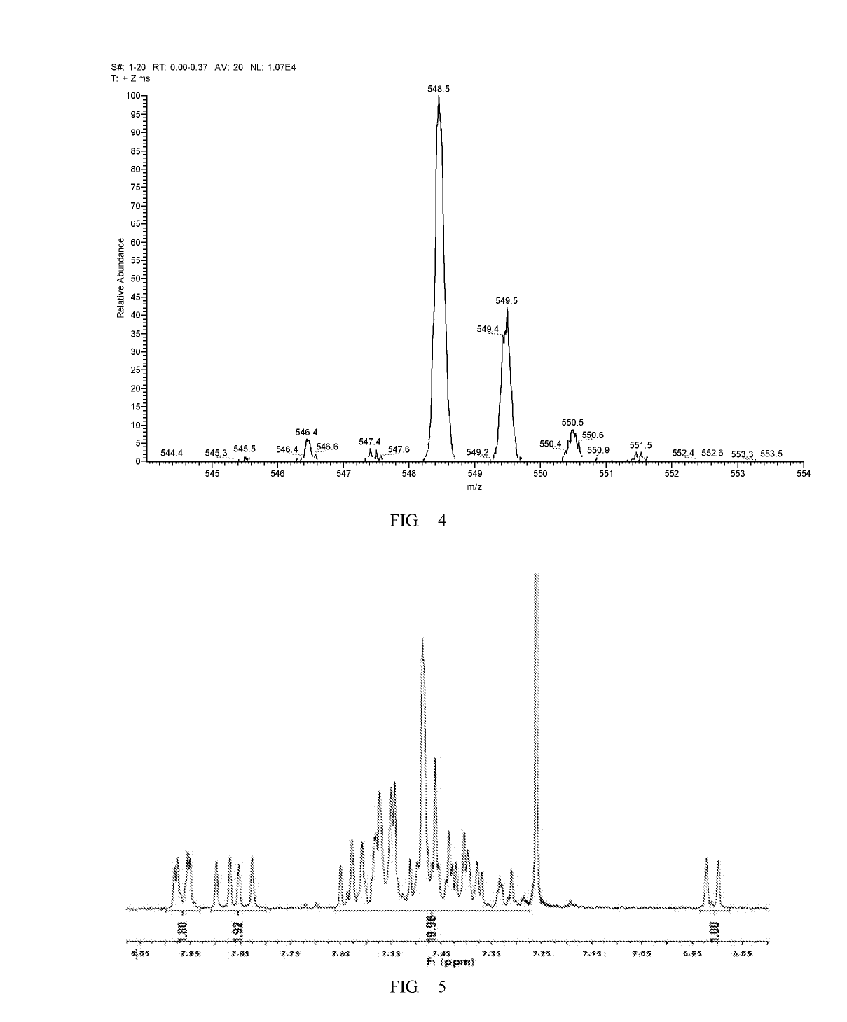

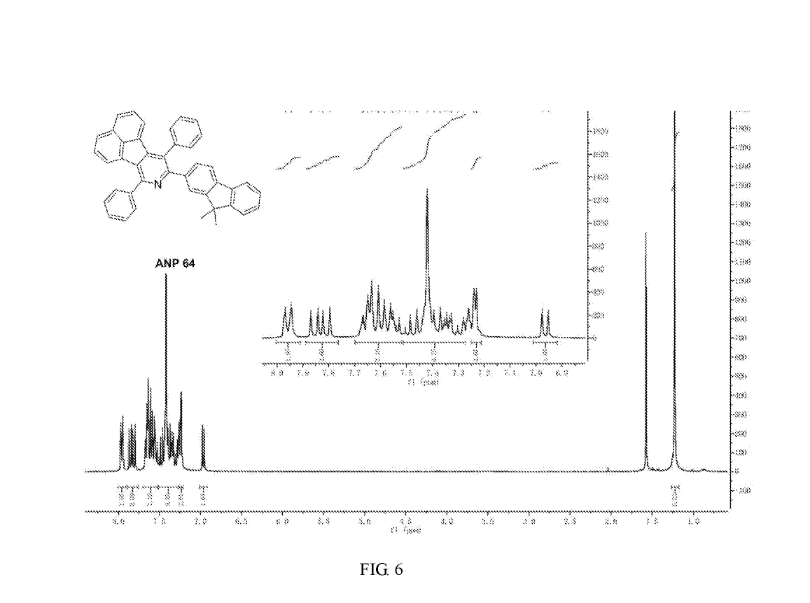

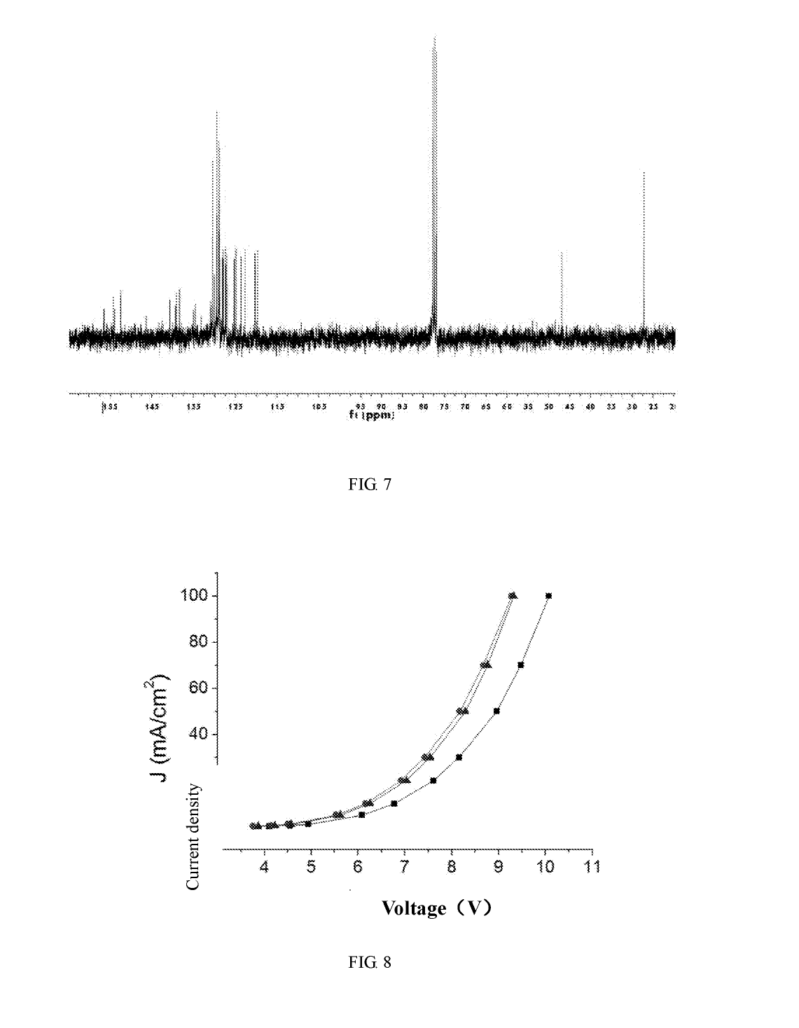

Under the nitrogen gas condition, mix the intermediate 8 (4.30 g, 5 mmol, Organic & Biomolecular Chemistry, 10(24), 4704-4711; 2012), 60 ml of diphenyl ether, heat them to reflux 48 hours (external temperature of 280° C.). Cool down the resulting brown solution to get the brown solid, and crystallize from dichloromethane-acetone to get 2.15 g of ANP 64 white crystal, with a yield of 50%. ¹H NMR (400 MHz, CDCl₃, δ): 7.98-7.94 (m, 2 H), 7.87-7.77 (m, 2 H), 7.67-7.22 (m, 18 H), 6.96 (d, 1 H, J=10 Hz), 1.23(s, 6 H), as shown in FIG. 6. ¹³C NMR (100 MHz, CDCl₃, δ): 27.8, 47.1, 119.6 120.3, 122.6, 123.6, 124.9, 125.3, 127.0, 127.3, 127.4, 127.9, 128.0, 128.1, 128.8, 129.0, 129.5, 130.0, 130.5, 130.9, 133.2, 134.6, as shown in FIG. 7. ESI-MS m/z value C₄₂H₂₉N: 547.23, actual measurement value [M+H]⁺: 548.53, as shown in FIG. 4.

### Embodiment 4

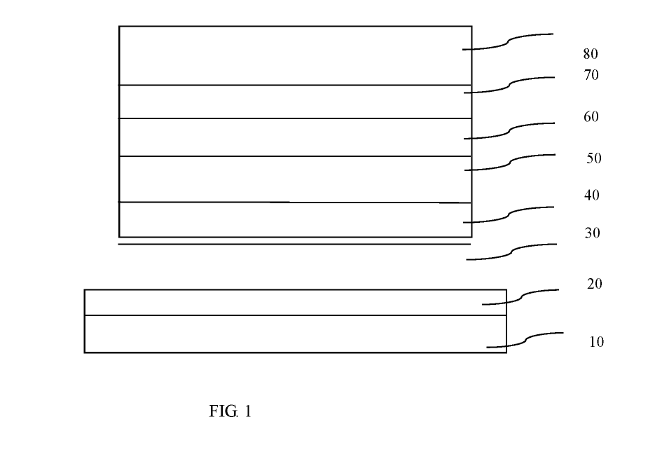

Produce OLED with the organic electroluminescent materials, with the device number 1. The structure of device is shown in FIG. 1.

Firstly, the ITO transparent conductive glass substrate 10 (with anode 20 above) is washed with detergent solution and deionized water, ethanol, acetone, deionized water in sequence, then treated with oxygen plasma for 30 seconds, and then treated with CFₓ plasma.

Then, perform vacuum evaporation of 75 nm NPB in ITO, which is used as the hole injection layer 30.

Then, perform vacuum evaporation of TCTA, to form 10 nm thick of hole transport layer 40.

Then, perform vacuum evaporation of 20 nm thick of ANP 34+1% compound 1 (the structure is shown below) in the hole transport layer as the light emitting layer 50.

Then, perform vacuum evaporation of 20 nm thick of compound BPhen in the light emitting layer, which is used as electron transport layer 60.

Finally, perform vacuum evaporation of 1 nm LiF as electron injection layer 70 and 100 nm Al cathode.

### Embodiment 5

The device number 2. The device structure is the same as that in Embodiment 4, except that the compound ANP 64 is used to replace the compound ANP 34.

### Embodiment 6

Produce OLED with the organic electroluminescent materials, with the device number 3. The structure of device is shown in FIG. 1.

Firstly, the ITO transparent conductive glass substrate 10 (with anode 20 above) is washed with detergent solution and deionized water, ethanol, acetone, deionized water in sequence, then treated with oxygen plasma for 30 seconds, and then treated with CFₓ plasma.

Then, perform vacuum evaporation of 60 nm 2-TNATA in ITO, which is used as the hole injection layer 30.

Then, perform vacuum evaporation of NPB, to form 10 nm thick of hole transport layer 40.

Then, perform vacuum evaporation of 30 nm thick of MADN in the hole transport layer as the light emitting layer 50.

Then, perform vacuum evaporation of 30 nm thick of ANP 34 in the light emitting layer, which is used as electron transport layer 60.

Finally, perform vacuum evaporation of 1 nm LiF as electron injection layer 70 and 100 nm Al cathode.

### Embodiment 7

The device number 4, the device structure is the same as that in the Embodiment 6, but the compound ANP 34 is replaced by ANP 64.

### Comparison Embodiment 1

The device number 5. The device is made according to the method in Embodiment 6, of which, the electron transport layer 60 of compound ANP 34 is replaced by Alq₃.

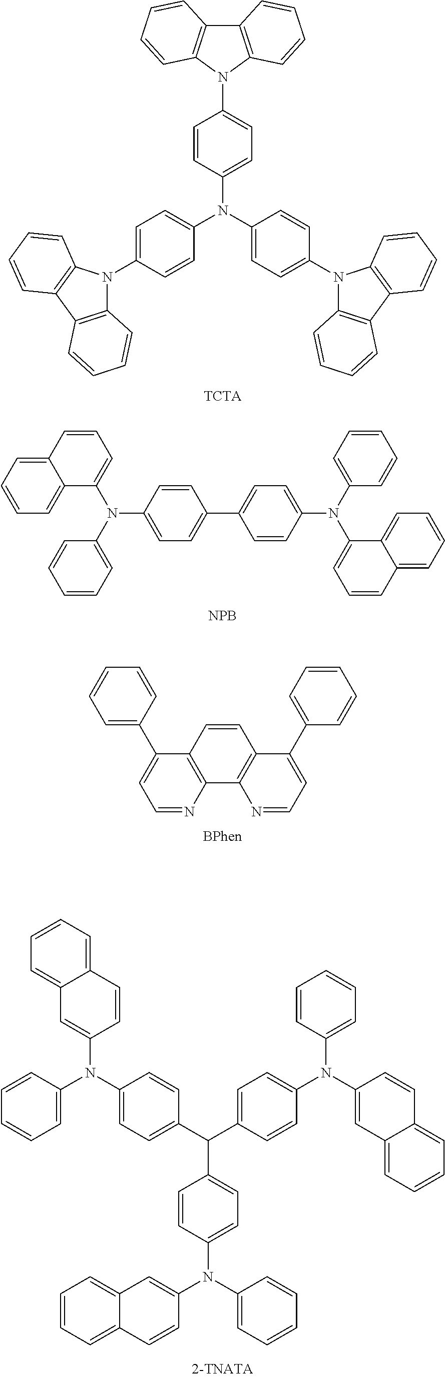 [SMILES: c1ccc2c(c1)c1ccccc1n2-c1ccc(N(c2ccc(-n3c4ccccc4c4ccccc43)cc2)c2ccc(-n3c4ccccc4c4ccccc43)cc2)cc1 ; c1ccc(N(c2ccc(-c3ccc(N(c4ccccc4)c4cccc5ccccc45)cc3)cc2)c2cccc3ccccc23)cc1 ; "NPB" ; c1ccc(-c2ccnc3c2ccc2c(-c4ccccc4)ccnc23)cc1 ; c1ccc(N(c2ccc(C(c3ccc(N(c4ccccc4)c4ccc5ccccc5c4)cc3)c3ccc(N(c4ccccc4)c4ccc5ccccc5c4)cc3)cc2)c2ccc3ccccc3c2)cc1]

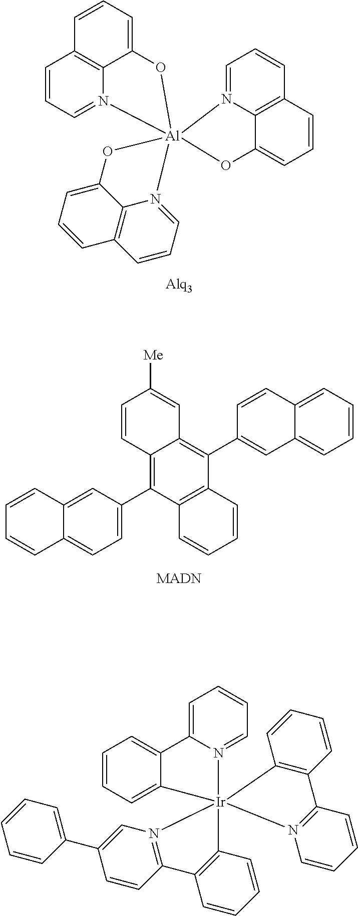

Compound 1

The parameters of the device under the current density of 20 mA/cm² are shown in the table I:

| | Volt- | | Current | Luminous | | |
|---|---|---|---|---|---|---|
| Device | age | Luminance | efficiency | efficiency | | |
| No. | V | cd/m² | cd/A | lm/W | CIEx | CIEy |
| 1 | 5.51 | 79.7 | 0.398 | 0.227 | 0.2285 | 0.3592 |
| 2 | 4.95 | 54.4 | 0.27 | 0.17 | 0.1915 | 0.32 |
| 3 | 7.04 | 380 | 1.90 | 0.847 | 0.184 | 0.232 |
| 4 | 6.92 | 265 | 1.32 | 0.599 | 0.168 | 0.249 |
| 5 | 7.61 | 341 | 1.71 | 0.706 | 0.170 | 0.241 |

As shown from the above table, the OED has good performance when the compound with ANP group is used as the electron transport (devices 1 and 2) or host material (components 3 and 4). As shown from the V-J curve in FIG. 8, the devices 3 and 4 have lower drive voltage (the drive voltage of device 5 under the current density of 20 mA/cm2 is 7.61V) compared with the device 5, demonstrating that the compound containing ANP group may be used as the host material or electron transport material of phosphorescent OED.

What is claimed is:

1. An organic electroluminescent material having the structure of formula (I) as described below: 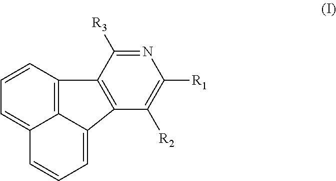
wherein, R₁-R₃ independently represent hydrogen, deuterium atom, halogen, hydroxy, cyano, nitro, amino, C1-C20 alkyl, C1-C20 alkoxy, C6-C40 aryl containing one or more substituents R or unsubstituted aryl, C6-C40 aromatic hydrocarbon group, C3-C40 aryl containing one or more substituents R or unsubstituted aryl containing one ore more hetero atoms, trialkylsilyl, triaryl silyl, trialkylsilyl containing one or more substituent R or unsubstituted trialkylsilyl, dicarboxylic phosphoroso containing one or more substituent R or unsubstituted diaryl phosphoroso, aryl carbonyl containing one or more substituent R or unsubstituted aryl carbonyl, diaryl amino containing one or more substituent R or unsubstituted diaryl amino, and the hetero atom is B, O, S, N, Se, the substituent R is halogen, hydroxyl, cyano, nitro, amino, C1-C4 alkyl, C1-C4 alkoxy;

2. The organic electroluminescent material according to claim 1, wherein R₂, R₃ are independently selected from hydrogen, halogen, C1-C8 alkyl, C6-C30 phenyl containing one or more substituent R or unsubstituted phenyl, C10-C30 fused aromatic ring group containing one or more substituent R or unsubstituted one, C6-C20 five- or six-membered heteroaryl containing one or more substituent R or unsubstituted heteroaryl containing one or two hetero atoms, C6-C30 diaromatic amino containing one or more substituent R or unsubstituted diaromatic amino; the substituent R is halogen, cyano, nitro, amino, C1-C4 alkyl, C1-C4 alkoxy, and the hetero atom is O, S, N.

3. The organic electroluminescent material according to claim 1, wherein R₂, R₃ are independently selected from hydrogen, halogen, C1-C4 alkyl, phenyl containing one substituent R or unsubstituted phenyl, naphthyl containing one substituent R or unsubstituted naphthyl, carbazolyl containing one substituent R or unsubstituted carbazolyl, five- or six-membered heteroaryl containing one hetero atom, and the substituents R is halogen, amino, C1-C4 alkyl.

4. The organic electroluminescent material according to claim 1, wherein R₂, R₃ can both be hydrogen, C1-C4 alkyl, phenyl, naphthyl, tolyl, thiophene furosemide, furan, pyrrole or pyrazine.

5. The organic electroluminescent material according to claim 1-4, wherein R₁ is selected from hydrogen, halogen, C1-C8 alkyl, C6-C20 five-or six-membered heteroaryl containing one or more substituent R or unsubstituted one containing one or more hetero atoms, C10-C20 fused aromatic ring group containing one or more substituent R or unsubstituted one, C6-C30 phenyl containing one or more substituent R or unsubstituted henyl, diphenyl amino, phenyl naphthylamino, triphenyl silyl, diphenylphosphineoxide, phenylcarbonyl or phenylsulfenyl, the substituent R is halogen, cyano, nitro, amino, C1-C4 alkyl, C1-C4 alkoxy, and the heteroatom is O, S, N.

6. The organic electroluminescent material according to claim 5, wherein R₁ is selected from hydrogen, halogen, C1-C4 alkyl, C10-C20 carbazolyl containing one substituent R or unsubstituted carbazolyl, C10-C20 fluorenyl containing one substituent R or unsubstituted fluorenyl, naphthyl, phenyl, C6-C10 five- or six-membered heteroaryl containing one or more substituent R or unsubstituted one.

7. The organic electroluminescent material according to claim 6, wherein the five- or six-membered heteroaryl containing one or more hetero atoms is pyrimidinyl, pyridyl, thiazolyl, triazole or triazinyl, the fluorenyl containing one or more substituent R or unsubstituted fluorenyl is 9,9-dimethyl-fluorenyl, 9,9-diphenyl fluorenyl, 9,9-xylyl fluorenyl or spirofluorenyl.

8. The organic electroluminescent material according to claim 7, wherein R₂, R₃ are both phenyl, R₁ is phenyl, bisbiphenyl, naphthyl, carbazolyl substituted by one substituent R, or R₁ is 9,9-dimethyl fluorenyl, 9,9 diphenyl fluorenyl, 9,9-xylyl fluorenyl or spirofluorenyl, and the substituent R is halogen, amino, C1-C4 alkyl.

9. The organic electroluminescent material according to claim 1, it is the following compounds:

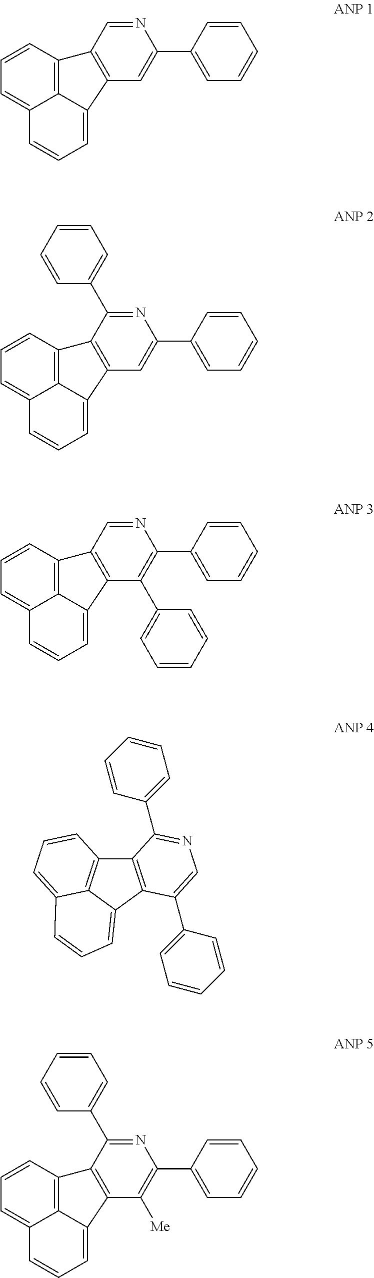 [SMILES: c1ccc(-c2cc3c(cn2)-c2cccc4cccc-3c24)cc1 ; c1ccc(-c2cc3c(c(-c4ccccc4)n2)-c2cccc4cccc-3c24)cc1 ; c1ccc(-c2ncc3c(c2-c2ccccc2)-c2cccc4cccc-3c24)cc1 ; c1ccc(-c2cnc(-c3ccccc3)c3c2-c2cccc4cccc-3c24)cc1 ; Cc1c(-c2ccccc2)nc(-c2ccccc2)c2c1-c1cccc3cccc-2c13]

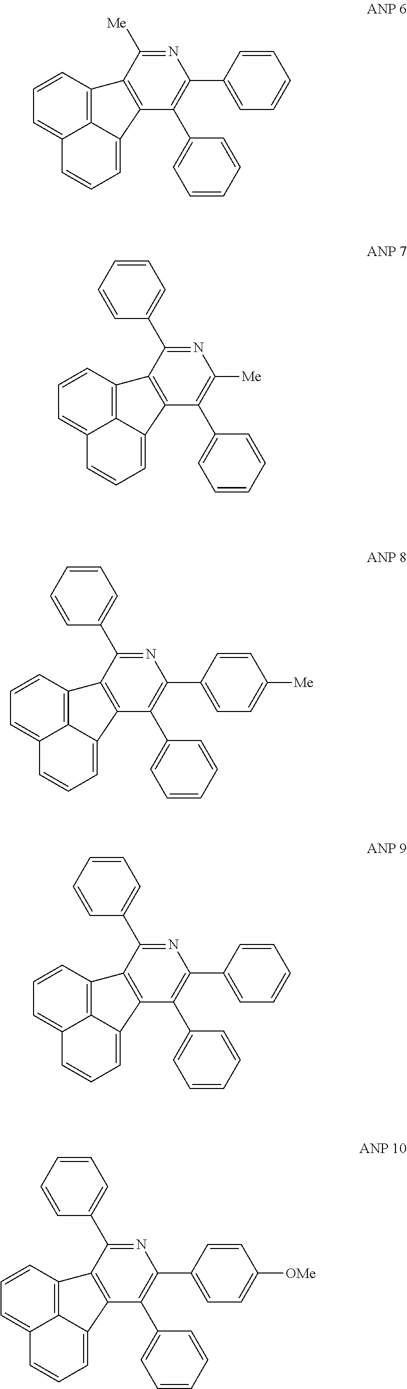 [SMILES: Cc1nc(-c2ccccc2)c(-c2ccccc2)c2c1-c1cccc3cccc-2c13 ; Cc1nc(-c2ccccc2)c2c(c1-c1ccccc1)-c1cccc3cccc-2c13 ; Cc1ccc(-c2nc(-c3ccccc3)c3c(c2-c2ccccc2)-c2cccc4cccc-3c24)cc1 ; c1ccc(-c2nc(-c3ccccc3)c3c(c2-c2ccccc2)-c2cccc4cccc-3c24)cc1 ; COc1ccc(-c2nc(-c3ccccc3)c3c(c2-c2ccccc2)-c2cccc4cccc-3c24)cc1]

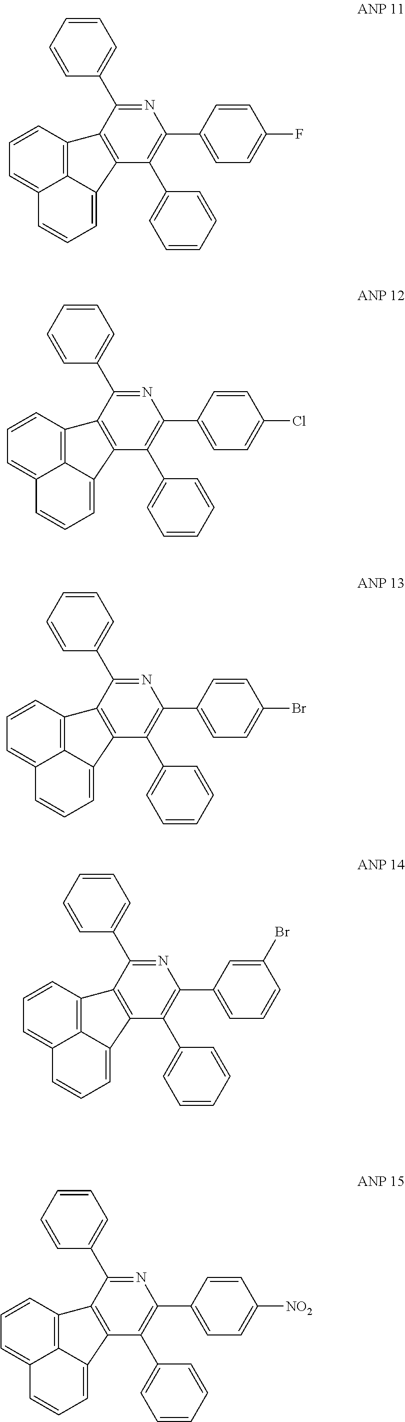 [SMILES: Fc1ccc(-c2nc(-c3ccccc3)c3c(c2-c2ccccc2)-c2cccc4cccc-3c24)cc1 ; Clc1ccc(-c2nc(-c3ccccc3)c3c(c2-c2ccccc2)-c2cccc4cccc-3c24)cc1 ; Brc1ccc(-c2nc(-c3ccccc3)c3c(c2-c2ccccc2)-c2cccc4cccc-3c24)cc1 ; Brc1cccc(-c2nc(-c3ccccc3)c3c(c2-c2ccccc2)-c2cccc4cccc-3c24)c1 ; O=[N+]([O-])c1ccc(-c2nc(-c3ccccc3)c3c(c2-c2ccccc2)-c2cccc4cccc-3c24)cc1]

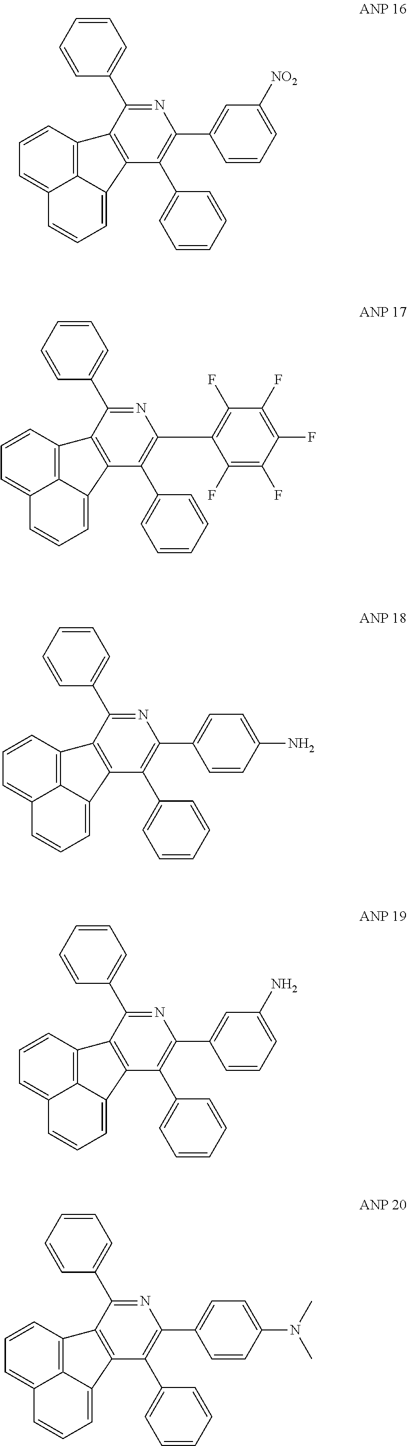 [SMILES: O=[N+]([O-])c1cccc(-c2nc(-c3ccccc3)c3c(c2-c2ccccc2)-c2cccc4cccc-3c24)c1 ; Fc1c(F)c(F)c(-c2nc(-c3ccccc3)c3c(c2-c2ccccc2)-c2cccc4cccc-3c24)c(F)c1F ; Nc1ccc(-c2nc(-c3ccccc3)c3c(c2-c2ccccc2)-c2cccc4cccc-3c24)cc1 ; Nc1cccc(-c2nc(-c3ccccc3)c3c(c2-c2ccccc2)-c2cccc4cccc-3c24)c1 ; CN(C)c1ccc(-c2nc(-c3ccccc3)c3c(c2-c2ccccc2)-c2cccc4cccc-3c24)cc1]

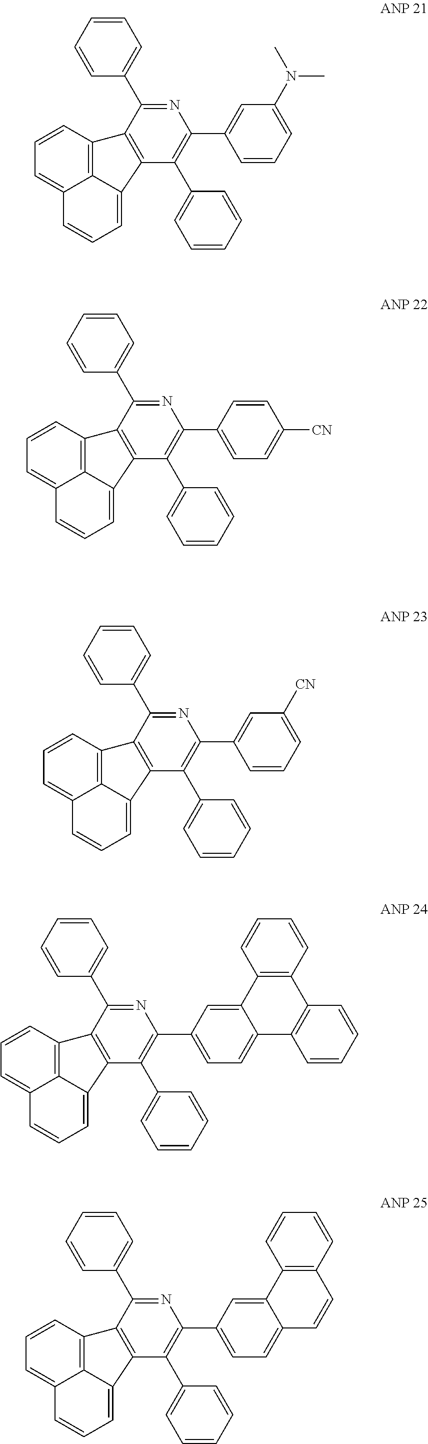 [SMILES: CN(C)c1cccc(-c2nc(-c3ccccc3)c3c(c2-c2ccccc2)-c2cccc4cccc-3c24)c1 ; N#Cc1ccc(-c2nc(-c3ccccc3)c3c(c2-c2ccccc2)-c2cccc4cccc-3c24)cc1 ; N#Cc1cccc(-c2nc(-c3ccccc3)c3c(c2-c2ccccc2)-c2cccc4cccc-3c24)c1 ; c1ccc(-c2nc(-c3ccc4c5ccccc5c5ccccc5c4c3)c(-c3ccccc3)c3c2-c2cccc4cccc-3c24)cc1 ; c1ccc(-c2nc(-c3ccc4ccc5ccccc5c4c3)c(-c3ccccc3)c3c2-c2cccc4cccc-3c24)cc1]

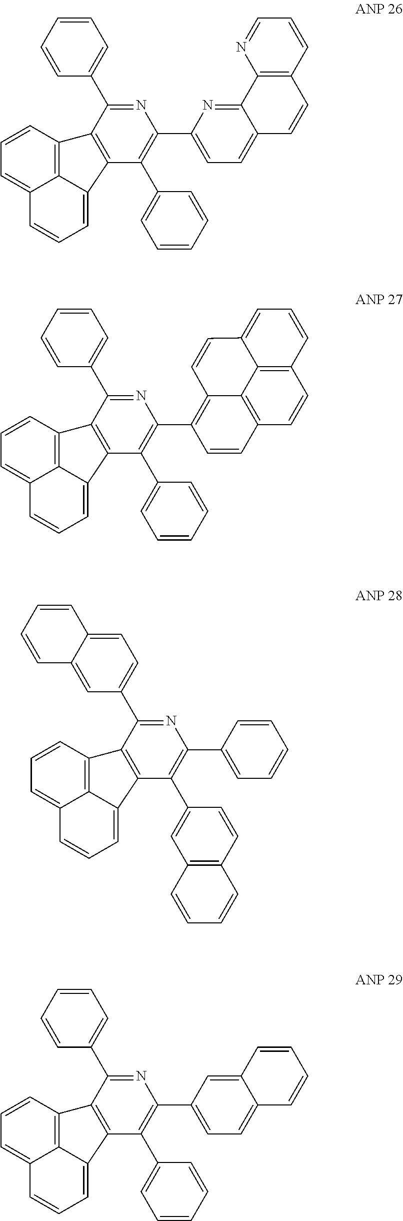 [SMILES: c1ccc(-c2nc(-c3ccc4ccc5cccnc5c4n3)c(-c3ccccc3)c3c2-c2cccc4cccc-3c24)cc1 ; c1ccc(-c2nc(-c3ccc4ccc5cccc6ccc3c4c56)c(-c3ccccc3)c3c2-c2cccc4cccc-3c24)cc1 ; c1ccc(-c2nc(-c3ccc4ccccc4c3)c3c(c2-c2ccc4ccccc4c2)-c2cccc4cccc-3c24)cc1 ; c1ccc(-c2nc(-c3ccc4ccccc4c3)c(-c3ccccc3)c3c2-c2cccc4cccc-3c24)cc1]

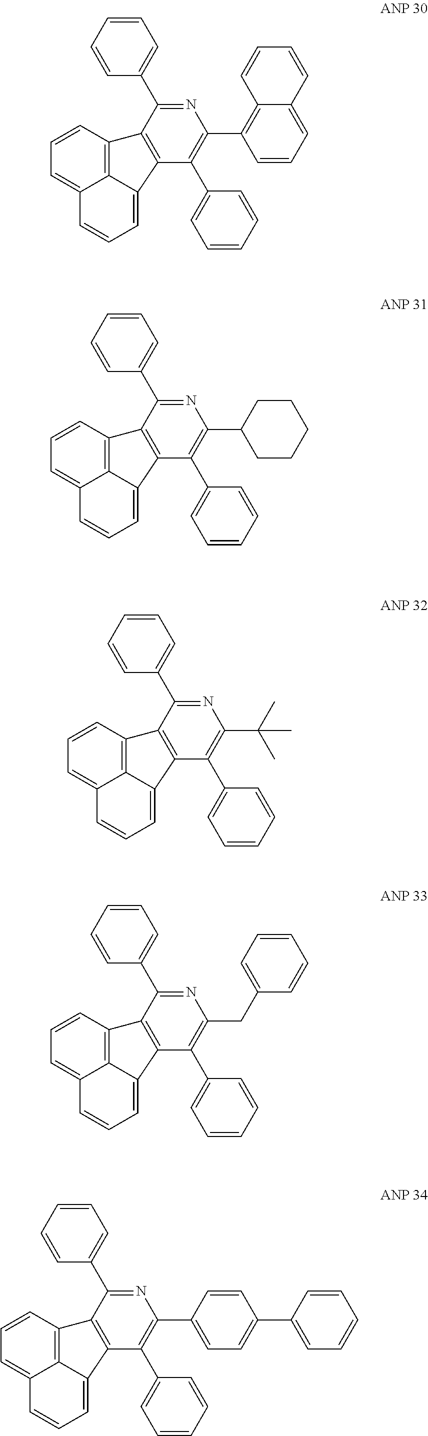 [SMILES: c1ccc(-c2nc(-c3cccc4ccccc34)c(-c3ccccc3)c3c2-c2cccc4cccc-3c24)cc1 ; c1ccc(-c2nc(C3CCCCC3)c(-c3ccccc3)c3c2-c2cccc4cccc-3c24)cc1 ; CC(C)(C)c1nc(-c2ccccc2)c2c(c1-c1ccccc1)-c1cccc3cccc-2c13 ; c1ccc(Cc2nc(-c3ccccc3)c3c(c2-c2ccccc2)-c2cccc4cccc-3c24)cc1 ; c1ccc(-c2ccc(-c3nc(-c4ccccc4)c4c(c3-c3ccccc3)-c3cccc5cccc-4c35)cc2)cc1]

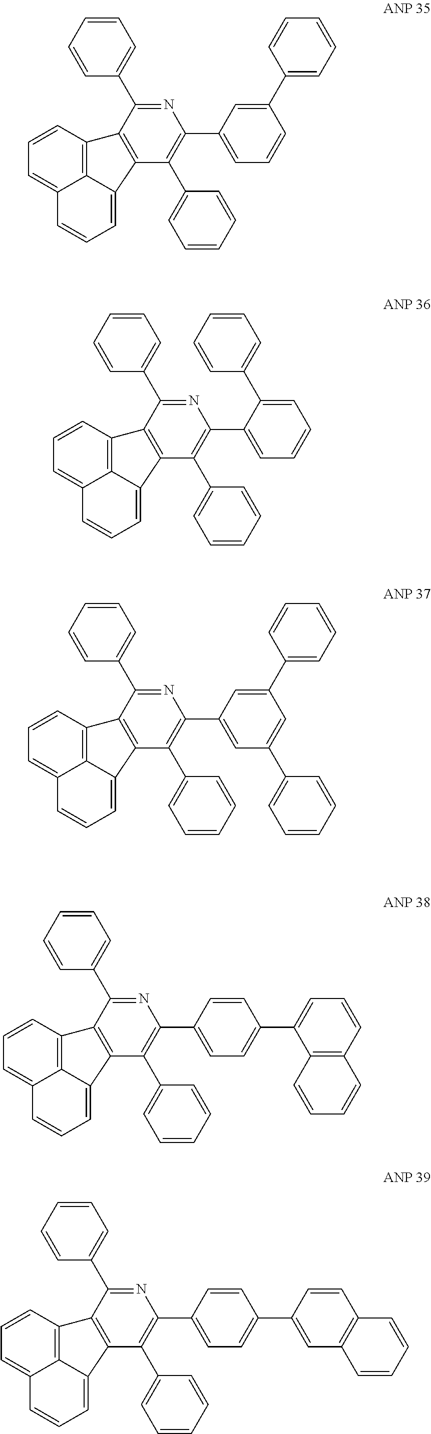 [SMILES: c1ccc(-c2cccc(-c3nc(-c4ccccc4)c4c(c3-c3ccccc3)-c3cccc5cccc-4c35)c2)cc1 ; c1ccc(-c2ccccc2-c2nc(-c3ccccc3)c3c(c2-c2ccccc2)-c2cccc4cccc-3c24)cc1 ; c1ccc(-c2cc(-c3ccccc3)cc(-c3nc(-c4ccccc4)c4c(c3-c3ccccc3)-c3cccc5cccc-4c35)c2)cc1 ; c1ccc(-c2nc(-c3ccc(-c4cccc5ccccc45)cc3)c(-c3ccccc3)c3c2-c2cccc4cccc-3c24)cc1 ; c1ccc(-c2nc(-c3ccc(-c4ccc5ccccc5c4)cc3)c(-c3ccccc3)c3c2-c2cccc4cccc-3c24)cc1]

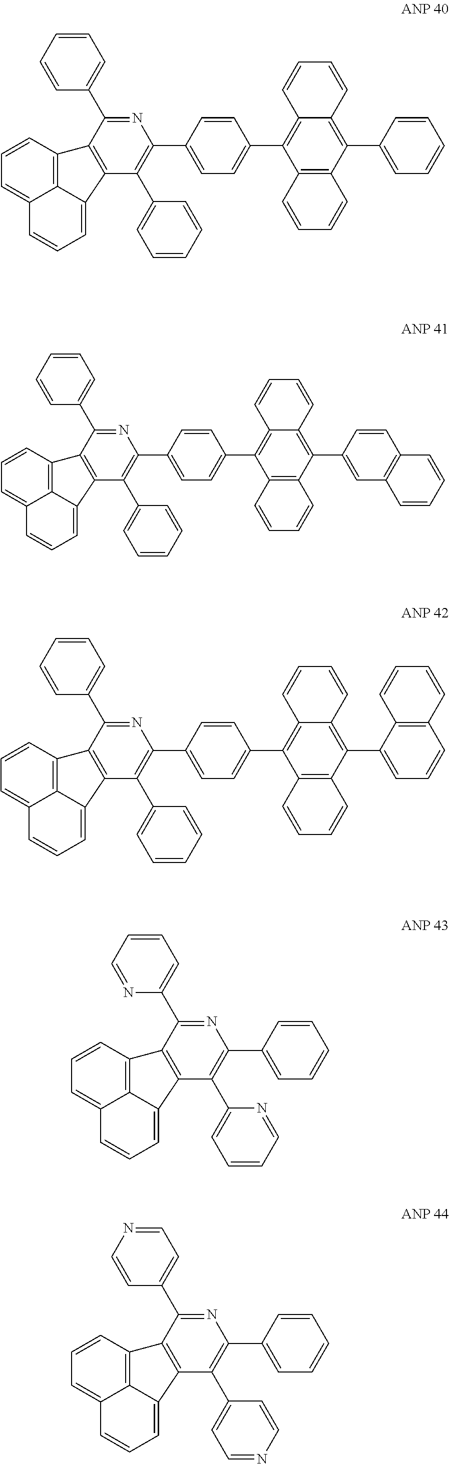 [SMILES: c1ccc(-c2nc(-c3ccc(-c4c5ccccc5c(-c5ccccc5)c5ccccc45)cc3)c(-c3ccccc3)c3c2-c2cccc4cccc-3c24)cc1 ; c1ccc(-c2nc(-c3ccc(-c4c5ccccc5c(-c5ccc6ccccc6c5)c5ccccc45)cc3)c(-c3ccccc3)c3c2-c2cccc4cccc-3c24)cc1 ; c1ccc(-c2nc(-c3ccc(-c4c5ccccc5c(-c5cccc6ccccc56)c5ccccc45)cc3)c(-c3ccccc3)c3c2-c2cccc4cccc-3c24)cc1 ; c1ccc(-c2nc(-c3ccccn3)c3c(c2-c2ccccn2)-c2cccc4cccc-3c24)cc1 ; c1ccc(-c2nc(-c3ccncc3)c3c(c2-c2ccncc2)-c2cccc4cccc-3c24)cc1]

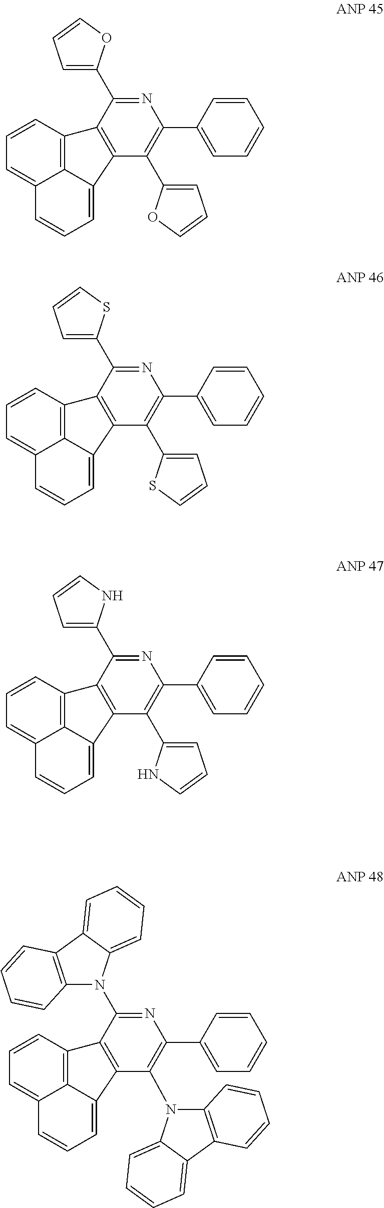 [SMILES: c1ccc(-c2nc(-c3ccco3)c3c(c2-c2ccco2)-c2cccc4cccc-3c24)cc1 ; c1ccc(-c2nc(-c3cccs3)c3c(c2-c2cccs2)-c2cccc4cccc-3c24)cc1 ; c1ccc(-c2nc(-c3ccc[nH]3)c3c(c2-c2ccc[nH]2)-c2cccc4cccc-3c24)cc1 ; c1ccc(-c2nc(-n3c4ccccc4c4ccccc43)c3c(c2-n2c4ccccc4c4ccccc42)-c2cccc4cccc-3c24)cc1]

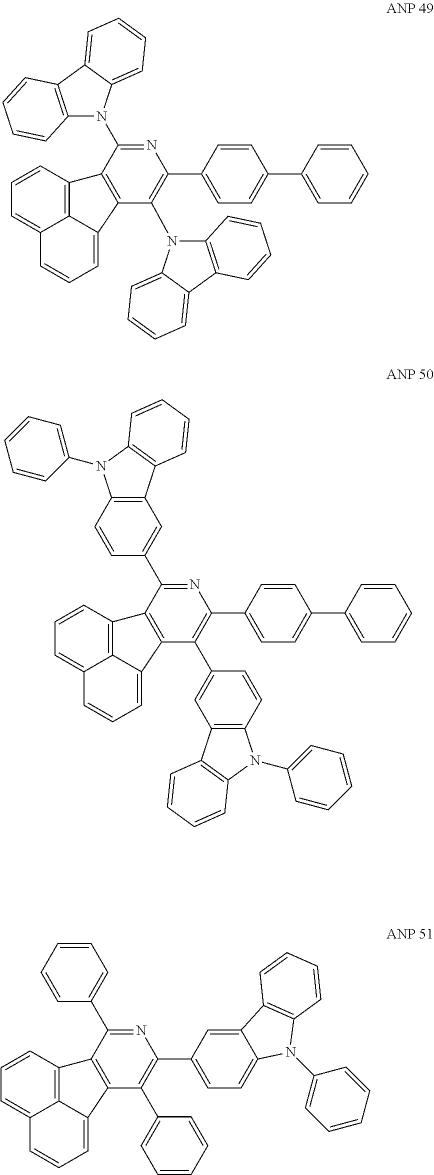 [SMILES: c1ccc(-c2ccc(-c3nc(-n4c5ccccc5c5ccccc54)c4c(c3-n3c5ccccc5c5ccccc53)-c3cccc5cccc-4c35)cc2)cc1 ; c1ccc(-c2ccc(-c3nc(-c4ccc5c(c4)c4ccccc4n5-c4ccccc4)c4c(c3-c3ccc5c(c3)c3ccccc3n5-c3ccccc3)-c3cccc5cccc-4c35)cc2)cc1 ; c1ccc(-c2nc(-c3ccc4c(c3)c3ccccc3n4-c3ccccc3)c(-c3ccccc3)c3c2-c2cccc4cccc-3c24)cc1]

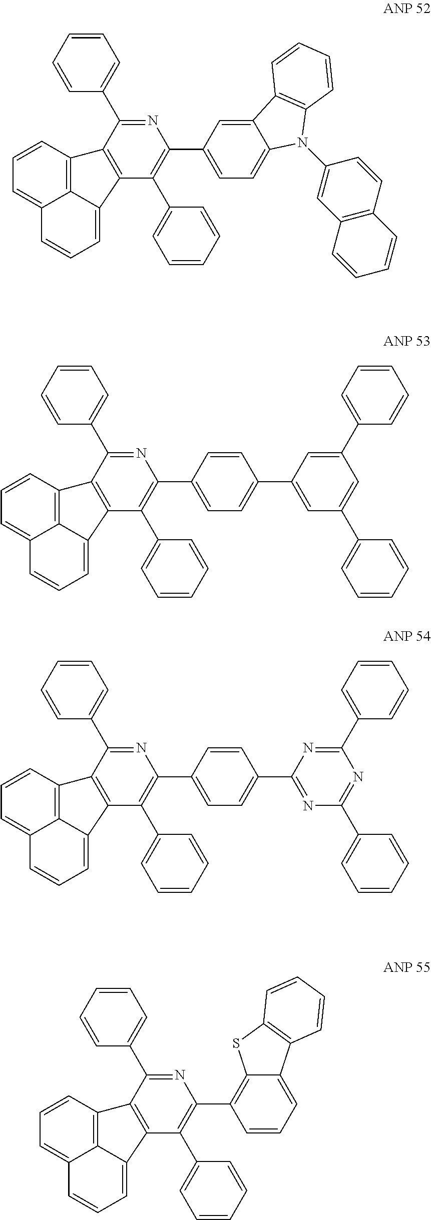 [SMILES: c1ccc(-c2nc(-c3ccc4c(c3)c3ccccc3n4-c3ccc4ccccc4c3)c(-c3ccccc3)c3c2-c2cccc4cccc-3c24)cc1 ; c1ccc(-c2cc(-c3ccccc3)cc(-c3ccc(-c4nc(-c5ccccc5)c5c(c4-c4ccccc4)-c4cccc6cccc-5c46)cc3)c2)cc1 ; c1ccc(-c2nc(-c3ccccc3)nc(-c3ccc(-c4nc(-c5ccccc5)c5c(c4-c4ccccc4)-c4cccc6cccc-5c46)cc3)n2)cc1 ; c1ccc(-c2nc(-c3cccc4c3sc3ccccc34)c(-c3ccccc3)c3c2-c2cccc4cccc-3c24)cc1]

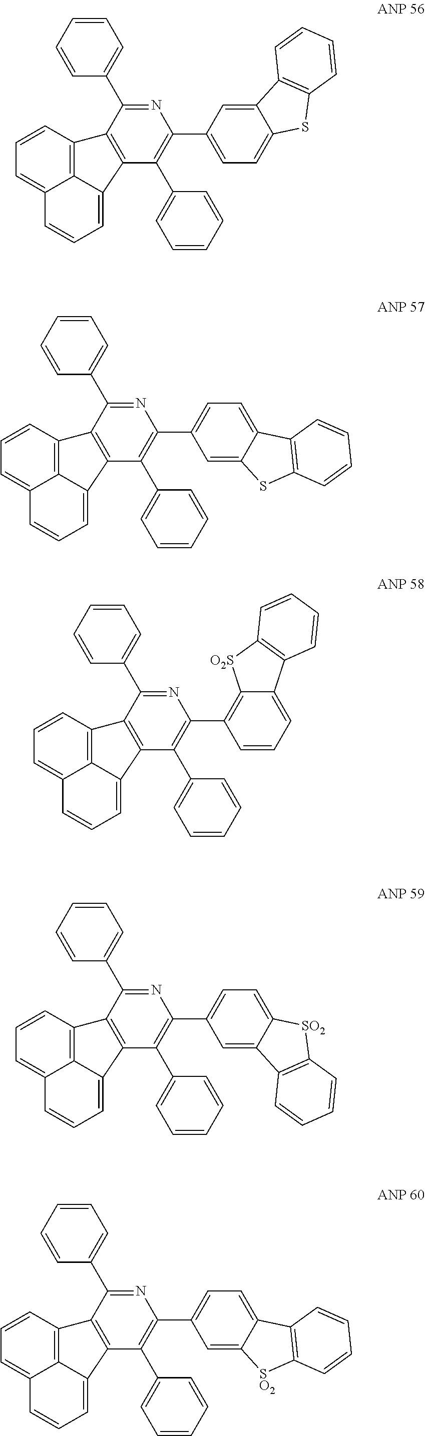 [SMILES: c1ccc(-c2nc(-c3ccc4sc5ccccc5c4c3)c(-c3ccccc3)c3c2-c2cccc4cccc-3c24)cc1 ; c1ccc(-c2nc(-c3ccc4c(c3)sc3ccccc34)c(-c3ccccc3)c3c2-c2cccc4cccc-3c24)cc1 ; O=S1(=O)c2ccccc2-c2cccc(-c3nc(-c4ccccc4)c4c(c3-c3ccccc3)-c3cccc5cccc-4c35)c21 ; O=S1(=O)c2ccccc2-c2cc(-c3nc(-c4ccccc4)c4c(c3-c3ccccc3)-c3cccc5cccc-4c35)ccc21 ; O=S1(=O)c2ccccc2-c2ccc(-c3nc(-c4ccccc4)c4c(c3-c3ccccc3)-c3cccc5cccc-4c35)cc21]

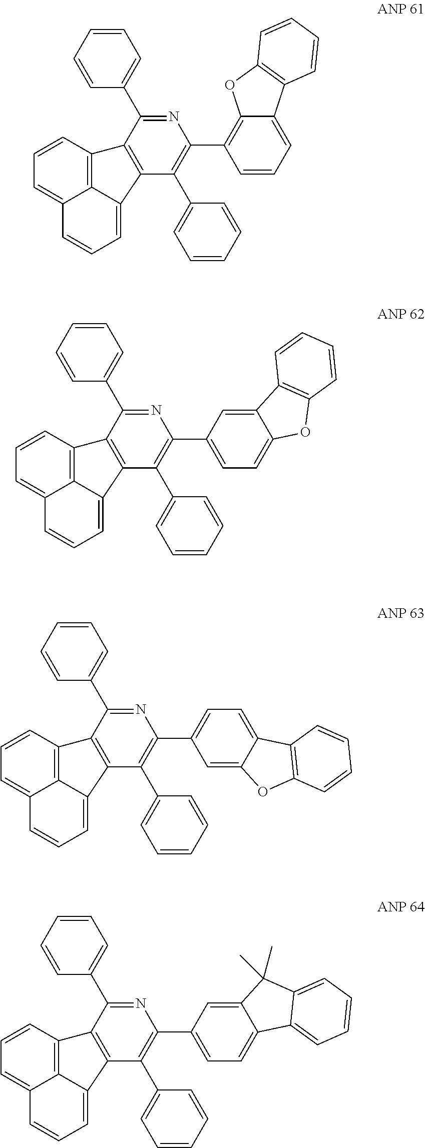 [SMILES: c1ccc(-c2nc(-c3cccc4c3oc3ccccc34)c(-c3ccccc3)c3c2-c2cccc4cccc-3c24)cc1 ; c1ccc(-c2nc(-c3ccc4oc5ccccc5c4c3)c(-c3ccccc3)c3c2-c2cccc4cccc-3c24)cc1 ; c1ccc(-c2nc(-c3ccc4c(c3)oc3ccccc34)c(-c3ccccc3)c3c2-c2cccc4cccc-3c24)cc1 ; CC1(C)c2ccccc2-c2ccc(-c3nc(-c4ccccc4)c4c(c3-c3ccccc3)-c3cccc5cccc-4c35)cc21]

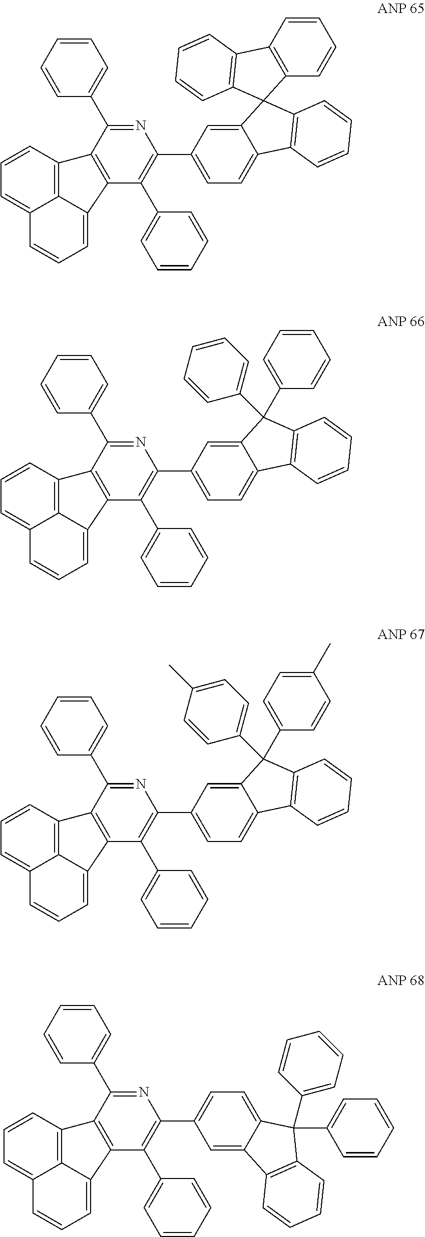 [SMILES: c1ccc(-c2nc(-c3ccc4c(c3)C3(c5ccccc5-c5ccccc53)c3ccccc3-4)c(-c3ccccc3)c3c2-c2cccc4cccc-3c24)cc1 ; c1ccc(-c2nc(-c3ccc4c(c3)C(c3ccccc3)(c3ccccc3)c3ccccc3-4)c(-c3ccccc3)c3c2-c2cccc4cccc-3c24)cc1 ; Cc1ccc(C2(c3ccc(C)cc3)c3ccccc3-c3ccc(-c4nc(-c5ccccc5)c5c(c4-c4ccccc4)-c4cccc6cccc-5c46)cc32)cc1 ; c1ccc(-c2nc(-c3ccc4c(c3)-c3ccccc3C4(c3ccccc3)c3ccccc3)c(-c3ccccc3)c3c2-c2cccc4cccc-3c24)cc1]

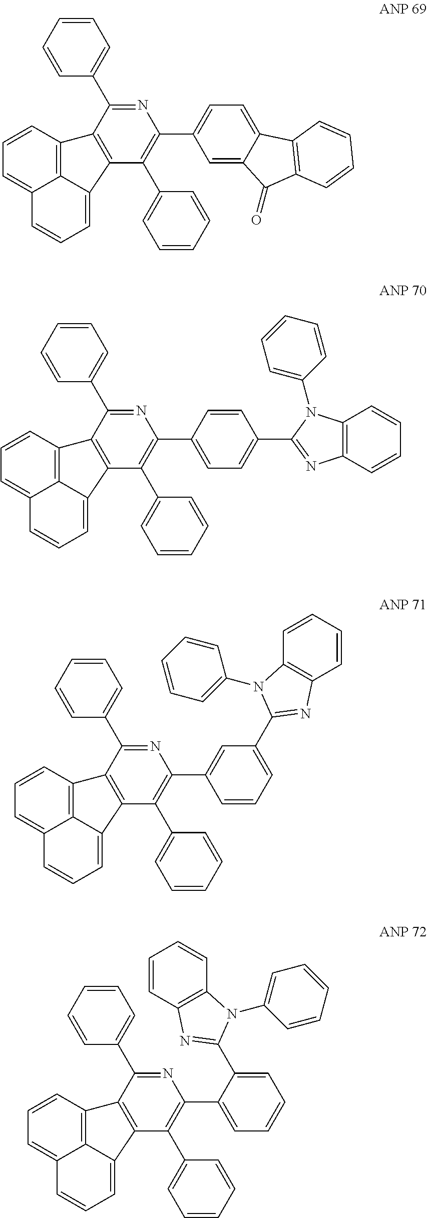 [SMILES: O=C1c2ccccc2-c2ccc(-c3nc(-c4ccccc4)c4c(c3-c3ccccc3)-c3cccc5cccc-4c35)cc21 ; c1ccc(-c2nc(-c3ccc(-c4nc5ccccc5n4-c4ccccc4)cc3)c(-c3ccccc3)c3c2-c2cccc4cccc-3c24)cc1 ; c1ccc(-c2nc(-c3cccc(-c4nc5ccccc5n4-c4ccccc4)c3)c(-c3ccccc3)c3c2-c2cccc4cccc-3c24)cc1 ; c1ccc(-c2nc(-c3ccccc3-c3nc4ccccc4n3-c3ccccc3)c(-c3ccccc3)c3c2-c2cccc4cccc-3c24)cc1]

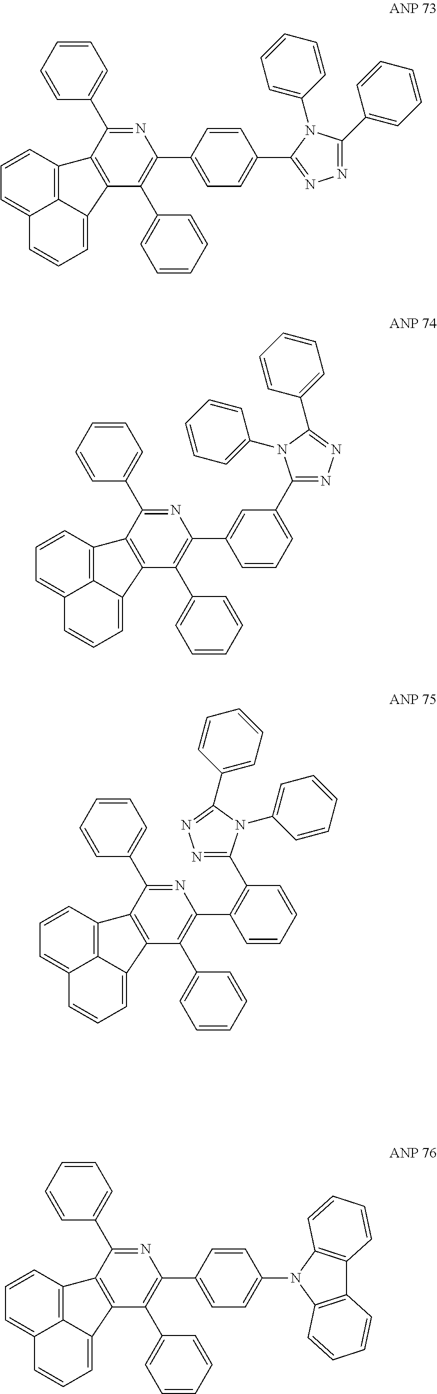 [SMILES: c1ccc(-c2nc(-c3ccc(-c4nnc(-c5ccccc5)n4-c4ccccc4)cc3)c(-c3ccccc3)c3c2-c2cccc4cccc-3c24)cc1 ; c1ccc(-c2nc(-c3cccc(-c4nnc(-c5ccccc5)n4-c4ccccc4)c3)c(-c3ccccc3)c3c2-c2cccc4cccc-3c24)cc1 ; c1ccc(-c2nc(-c3ccccc3-c3nnc(-c4ccccc4)n3-c3ccccc3)c(-c3ccccc3)c3c2-c2cccc4cccc-3c24)cc1 ; c1ccc(-c2nc(-c3ccc(-n4c5ccccc5c5ccccc54)cc3)c(-c3ccccc3)c3c2-c2cccc4cccc-3c24)cc1]

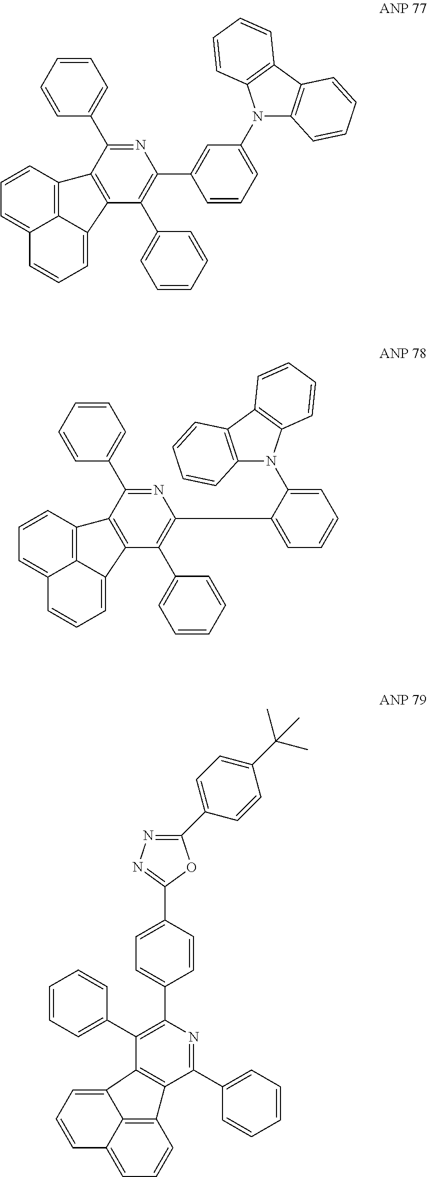 [SMILES: c1ccc(-c2nc(-c3cccc(-n4c5ccccc5c5ccccc54)c3)c(-c3ccccc3)c3c2-c2cccc4cccc-3c24)cc1 ; c1ccc(-c2nc(-c3ccccc3-n3c4ccccc4c4ccccc43)c(-c3ccccc3)c3c2-c2cccc4cccc-3c24)cc1 ; CC(C)(C)c1ccc(-c2nnc(-c3ccc(-c4nc(-c5ccccc5)c5c(c4-c4ccccc4)-c4cccc6cccc-5c46)cc3)o2)cc1]

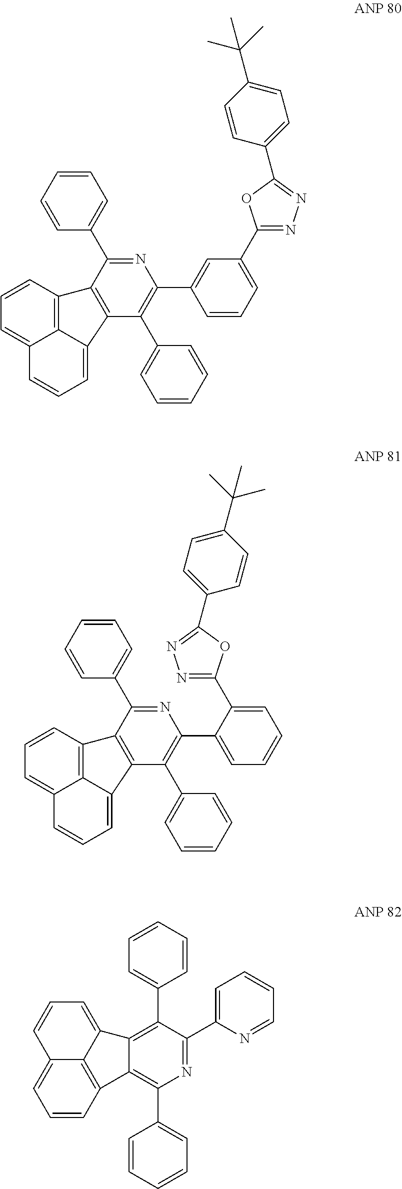 [SMILES: CC(C)(C)c1ccc(-c2nnc(-c3cccc(-c4nc(-c5ccccc5)c5c(c4-c4ccccc4)-c4cccc6cccc-5c46)c3)o2)cc1 ; CC(C)(C)c1ccc(-c2nnc(-c3ccccc3-c3nc(-c4ccccc4)c4c(c3-c3ccccc3)-c3cccc5cccc-4c35)o2)cc1 ; c1ccc(-c2nc(-c3ccccn3)c(-c3ccccc3)c3c2-c2cccc4cccc-3c24)cc1]

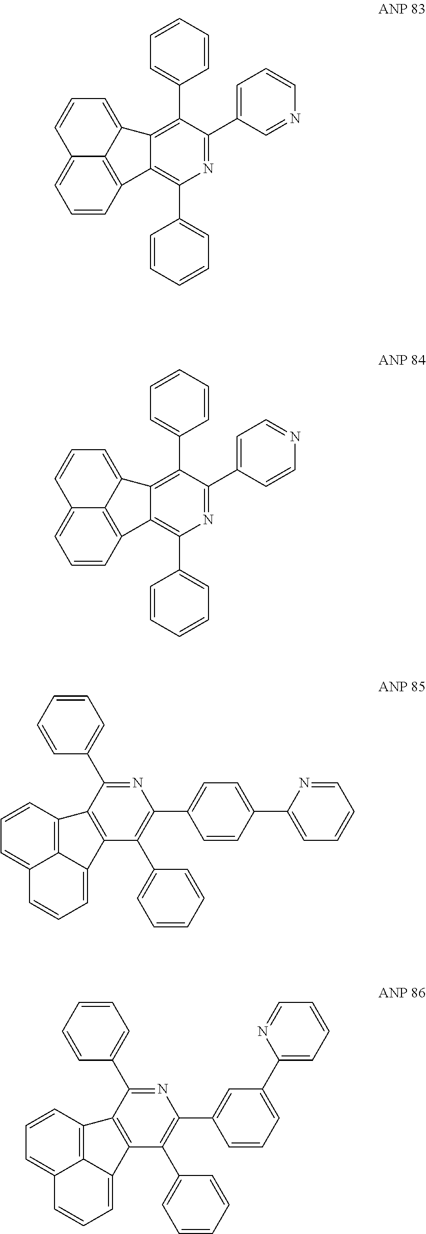 [SMILES: c1ccc(-c2nc(-c3cccnc3)c(-c3ccccc3)c3c2-c2cccc4cccc-3c24)cc1 ; c1ccc(-c2nc(-c3ccncc3)c(-c3ccccc3)c3c2-c2cccc4cccc-3c24)cc1 ; c1ccc(-c2nc(-c3ccc(-c4ccccn4)cc3)c(-c3ccccc3)c3c2-c2cccc4cccc-3c24)cc1 ; c1ccc(-c2nc(-c3cccc(-c4ccccn4)c3)c(-c3ccccc3)c3c2-c2cccc4cccc-3c24)cc1]

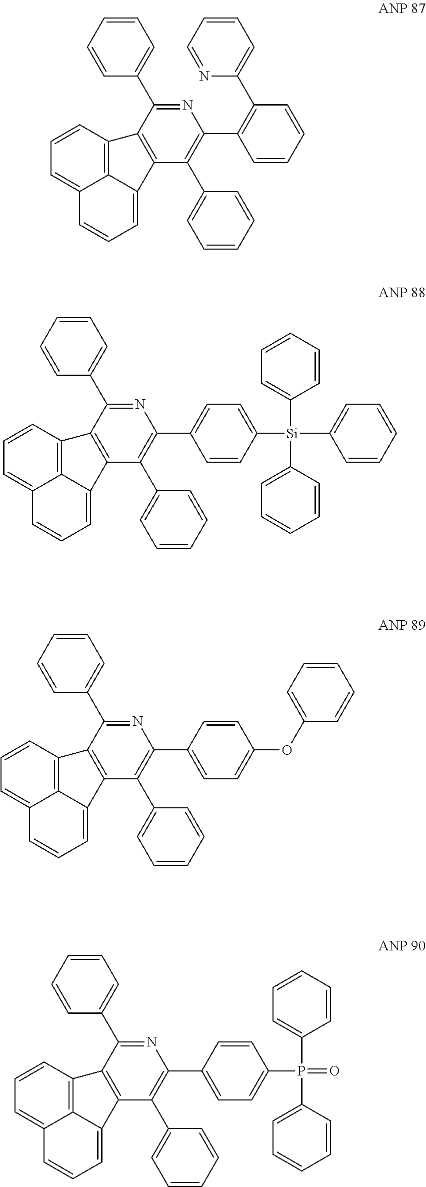 [SMILES: c1ccc(-c2nc(-c3ccccc3-c3ccccn3)c(-c3ccccc3)c3c2-c2cccc4cccc-3c24)cc1 ; c1ccc(-c2nc(-c3ccc([Si](c4ccccc4)(c4ccccc4)c4ccccc4)cc3)c(-c3ccccc3)c3c2-c2cccc4cccc-3c24)cc1 ; c1ccc(Oc2ccc(-c3nc(-c4ccccc4)c4c(c3-c3ccccc3)-c3cccc5cccc-4c35)cc2)cc1 ; O=P(c1ccccc1)(c1ccccc1)c1ccc(-c2nc(-c3ccccc3)c3c(c2-c2ccccc2)-c2cccc4cccc-3c24)cc1]

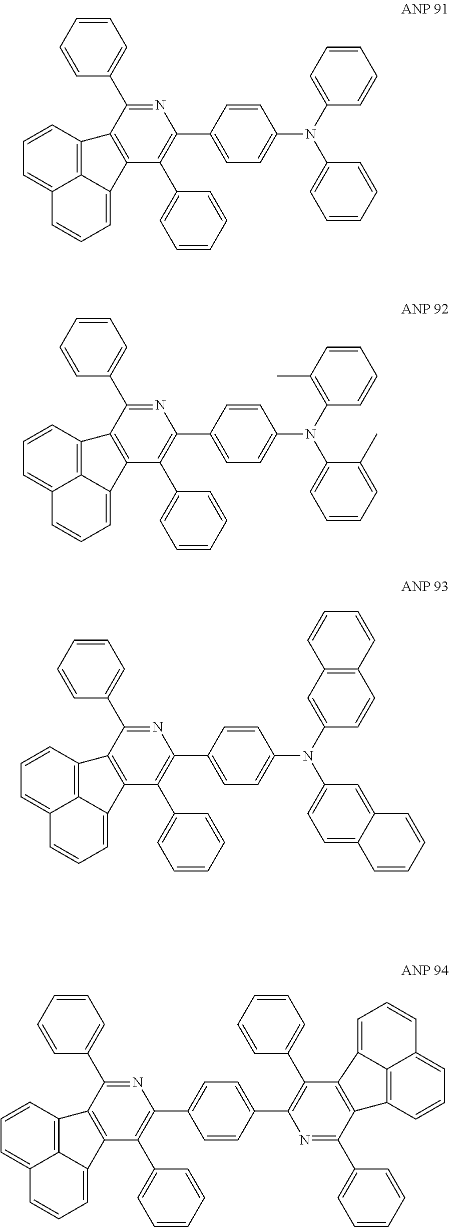 [SMILES: c1ccc(-c2nc(-c3ccc(N(c4ccccc4)c4ccccc4)cc3)c(-c3ccccc3)c3c2-c2cccc4cccc-3c24)cc1 ; Cc1ccccc1N(c1ccc(-c2nc(-c3ccccc3)c3c(c2-c2ccccc2)-c2cccc4cccc-3c24)cc1)c1ccccc1C ; c1ccc(-c2nc(-c3ccc(N(c4ccc5ccccc5c4)c4ccc5ccccc5c4)cc3)c(-c3ccccc3)c3c2-c2cccc4cccc-3c24)cc1 ; c1ccc(-c2nc(-c3ccc(-c4nc(-c5ccccc5)c5c(c4-c4ccccc4)-c4cccc6cccc-5c46)cc3)c(-c3ccccc3)c3c2-c2cccc4cccc-3c24)cc1]

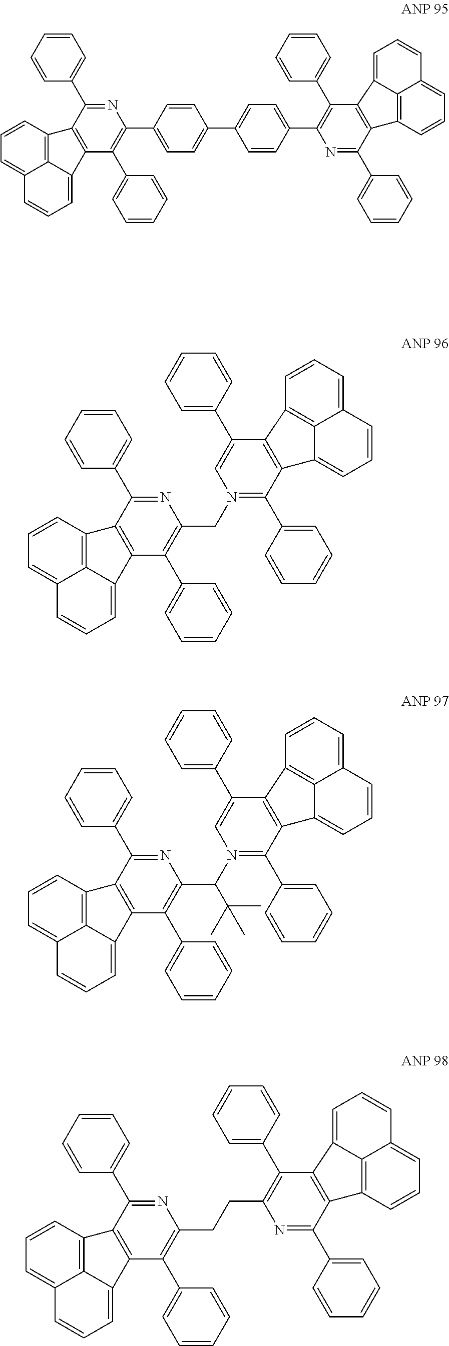

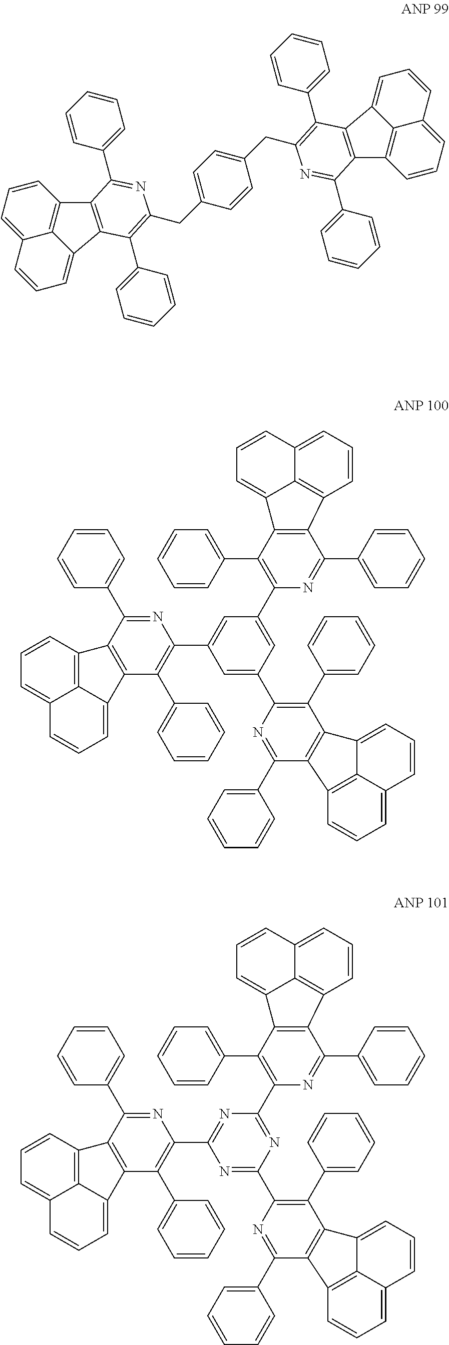 [SMILES: c1ccc(-c2nc(Cc3ccc(Cc4nc(-c5ccccc5)c5c(c4-c4ccccc4)-c4cccc6cccc-5c46)cc3)c(-c3ccccc3)c3c2-c2cccc4cccc-3c24)cc1 ; c1ccc(-c2nc(-c3cc(-c4nc(-c5ccccc5)c5c(c4-c4ccccc4)-c4cccc6cccc-5c46)cc(-c4nc(-c5ccccc5)c5c(c4-c4ccccc4)-c4cccc6cccc-5c46)c3)c(-c3ccccc3)c3c2-c2cccc4cccc-3c24)cc1 ; c1ccc(-c2nc(-c3nc(-c4nc(-c5ccccc5)c5c(c4-c4ccccc4)-c4cccc6cccc-5c46)nc(-c4nc(-c5ccccc5)c5c(c4-c4ccccc4)-c4cccc6cccc-5c46)n3)c(-c3ccccc3)c3c2-c2cccc4cccc-3c24)cc1]

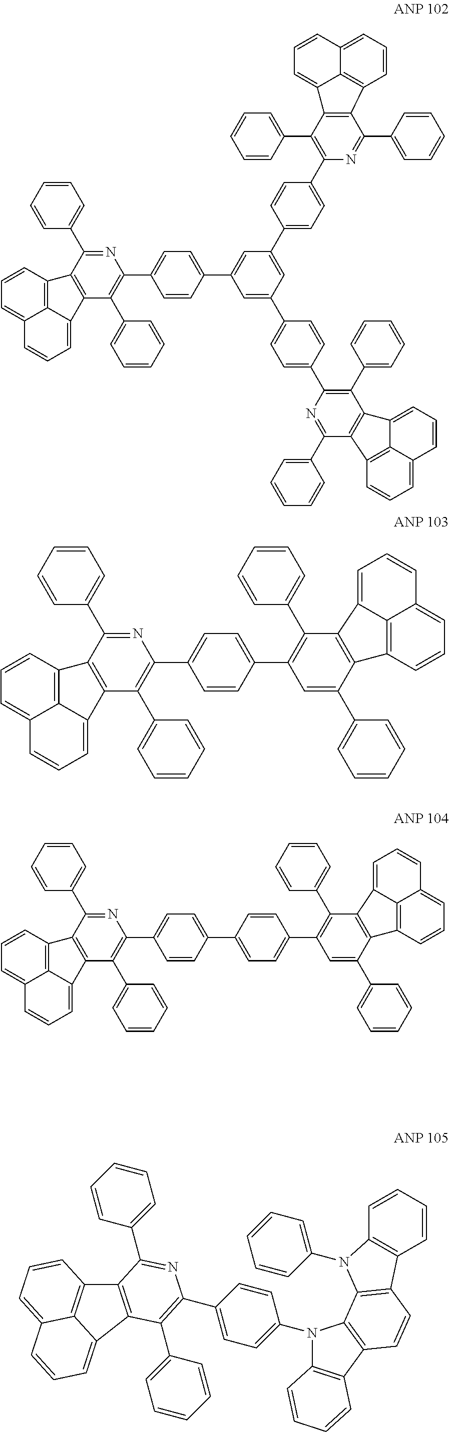

10. The organic electroluminescent material according to claim 1, it is the following compounds:

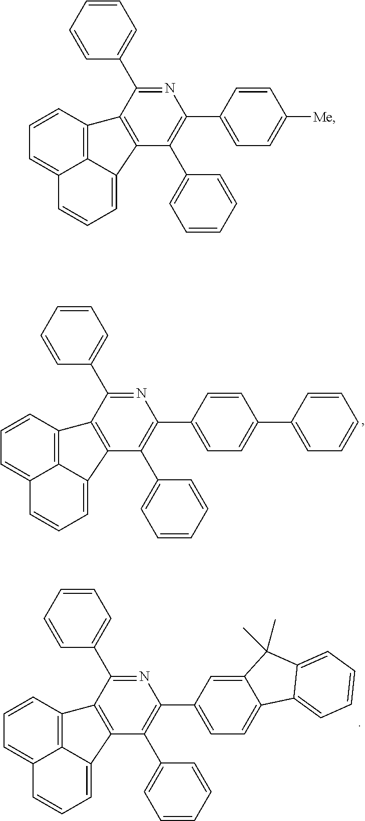 [SMILES: Cc1ccc(-c2nc(-c3ccccc3)c3c(c2-c2ccccc2)-c2cccc4cccc-3c24)cc1 ; c1ccc(-c2ccc(-c3nc(-c4ccccc4)c4c(c3-c3ccccc3)-c3cccc5cccc-4c35)cc2)cc1 ; CC1(C)c2ccccc2-c2ccc(-c3nc(-c4ccccc4)c4c(c3-c3ccccc3)-c3cccc5cccc-4c35)cc21]

11. The method for preparing organic electroluminescent material according to any one of claims 1-10, wherein the steps are as follows:
(1) Preparing 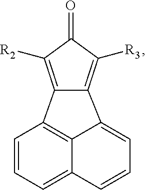
(2) reacting with R₁—CN for 40-50 hours under the temperature of 250-300° C. with the protection of nitrogen gas.

12. The preparation method according to claim 11, wherein the reaction in step (2) is to mix the raw materials for direct heating under the protection of nitrogen gas.

13. The preparation method according to claim 11, wherein the reaction in step (2) is to heat for reflux for 40-50 hours by adding solvent diphenyl ether.

14. The preparation method according to claim 11, wherein the step (2) further includes recrystallization purification: to recrystallize and purify with dichloromethane-acetone mixed solvent for the recrystallization.

15. The preparation method according to claim 14, wherein the silica gel column purification steps and petroleum ether eluting are included before the recrystallization.

16. The preparation method according to claim 11, wherein the method in the step (1):
reflux the acenaphthequinone and 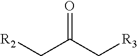
at 70-100° C. under the condition of nitrogen gas and strong alkaline condition.

17. The preparation method according to claim 14, wherein the strong alkaline condition is to add sodium hydroxide or potassium hydroxide in the solution, and the solvent of the reflux solution is ethanol.

18. An organic electroluminescent device containing the organic electroluminescent material as claimed in any one of claims 1-10.

19. The organic electroluminescent device according to claim 18, wherein the organic electroluminescent material as claimed in any one of claims 1-10 is used as the electron transporting material, and/or as the red phosphorescent host material in the light emitting layer.

20. The organic electroluminescent device according to claim 19, wherein the guest material is an organic iridium compound or an organic platinum compound.
